SEQUENCE LISTING (18 sequences)

SEQ ID NO: 1 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 2 - DNA, 1824 bp, Homo sapiens
  source 1..1824: mol_type = other DNA
gctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaa

SEQ ID NO: 3 - protein, 525 aa, Homo sapiens
MASNDYTQQATQSYGAYPTQPGQGYSQQSSQPYGQQSYSGYSQSTDTSGYGQSSYSSYGQSQNSYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 4 - protein, 522 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 5 - protein, 321 aa, Homo sapiens
MAIKTRVVEVAAVAMDSRTVEAAAGVAVVAAAAAAVVVTTAAVVAMNPEVVEVAVEAEVAWGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 6 - protein, 321 aa, Homo sapiens; identical to SEQ ID NO: 5

SEQ ID NO: 7 - protein, 524 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 8 - protein, 523 aa, Homo sapiens
MASNDYTQQATQSYGAYPTQPGQGYSQQSSQPYGQQSYSGYSQSTDTSGYGQSSYSSYGQSQNSYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 9 - RNA, 20 nt, synthetic construct
  source 1..20: mol_type = other RNA
tccacggacacttcaggcta

SEQ ID NO: 10 - RNA, 20 nt, unidentified
  source 1..20: mol_type = other RNA
tccacggacacttcaggata

SEQ ID NO: 11 - RNA, 20 nt, synthetic construct
  source 1..20: mol_type = other RNA
gggcagcagtcctcctaccc

SEQ ID NO: 12 - RNA, 20 nt, synthetic construct
  source 1..20: mol_type = other RNA
gggcagcagtcctcctatcc

SEQ ID NO: 13 - RNA, 20 nt, synthetic construct
  source 1..20: mol_type = other RNA
ctgctggccatagccagggt

SEQ ID NO: 14 - RNA, 20 nt, synthetic construct
  source 1..20: mol_type = other RNA
ctgctggccatagccaggat

SEQ ID NO: 15 - protein, 1368 aa, Streptococcus pyogenes [sequence omitted]

SEQ ID NO: 16 - DNA, 4101 bp, Streptococcus pyogenes
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 17 - protein, 1307 aa, Francisella novicida
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 18 - DNA, 3921 bp, Francisella novicida
  source 1..3921: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgcctccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgcgaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

(19) United States
(12) Patent Application Publication
(10) Pub. No.: US 2025/0002944 A1
(43) Pub. Date: Jan. 2, 2025

(54) ALLELE SPECIFIC EDITING TO TREAT FUS-INDUCED NEURODEGENERATION

(71) Applicant: The J. David Gladstone Institutes, a Testamentary Trust established under the Will of J. David Glad, San Francisco, CA (US)

(72) Inventors: Bruce Conklin, San Francisco, CA (US); Zachary Nevin, San Francisco, CA (US); Mengyuan Sun, San Francisco, CA (US); Madeline Matia, San Francisco, CA (US); Hannah Watry, San Francisco, CA (US)

(21) Appl. No.: 18/699,038

(22) PCT Filed: Oct. 5, 2022
(86) PCT No.: PCT/US2022/077616

§ 371 Date: Apr. 5, 2024

Related U.S. Application Data
(60) Provisional application No. 63/252,308, filed on Oct. 5, 2021.

(51) Int. Cl. C12N 15/90; C12N 9/22; C12N 15/11

(52) U.S. Cl. CPC C12N 15/907; C12N 9/22; C12N 15/11; C12N 2310/20; C12N 2320/34

(57) ABSTRACT

Methods and compositions are described herein that are useful to treat pathological mutation in the fused in sarcoma (FUS) gene. A number of FUS mutations are correlated with neurodegenerative conditions such as frontotemporal dementia (FTD) and amyotrophic lateral sclerosis (ALS). The methods and compositions can target and reduce or eliminate such FUS mutations.

### CROSS-REFERENCE TO RELATED APPLICATIONS

This application claims the benefit of priority to U.S. Provisional Ser. No. 63/252,308, filed Oct. 5, 2021, which is incorporated by reference as if fully set forth herein.

### GOVERNMENT SUPPORT

This invention was made with government support under R01-HL130533, R01-HL135358, R01-EY028249, RF1-AG072052, and U01-HL145795 awarded by the National Institutes of Health. The government has certain rights in the invention.

### INCORPORATION BY REFERENCE OF SEQUENCE LISTING PROVIDED AS AN XML FILE

A Sequence Listing is provided herewith as an xml file, “2275326.xml” created on Oct. 4, 2022 and having a size of 32,768 bytes. The content of the xml file is incorporated by reference herein in its entirety.

### BACKGROUND

Frontotemporal dementia (FTD) and amyotrophic lateral sclerosis (ALS) are fatal neurodegenerative diseases caused by loss of motoneurons (MNs) in the brain and spinal cord, leading to progressive muscle atrophy. Although FUS-FTD/ALS conditions are rare they are devastating and incurable.

Within the past ten years, mutations in the gene “fused in sarcoma” (FUS) have recently been recognized to cause approximately 5% of all genetic cases of FTD and ALS, collectively termed FUS-FTD/ALS. Other genes that are commonly involved in ALS include Cu/Zn superoxide dismutase 1 (SOD1), C9orf72, and Tar DNA-binding protein 43 (TDP-43). However, a clear correlation between various genetic defects and the molecular causes of neuron dysfunction has currently not been established.

In addition, there are over 60 different FUS mutations that are linked to disease. However, many mutations are not targetable by CRISPR guide RNAs, and frame-shift mutations in the last two exons of FUS result in disease-causing protein truncations, rendering mutation-specific editing in this region essentially futile.

### SUMMARY

Methods and compositions are described herein provide a solution to the CRISPR editing problems by targeting FUS loci linked upstream (5′) to disease-related FU/S mutations. Editing each FUS mutation individually is impractical. By targeting FUS exons upstream of many FUS mutations in one allele, the deleterious effects of those numerous FUS mutations are removed.

Hence, the methods and compositions involve editing one endogenous mutant FUS allele to inactivate that mutant allele and reduce or eliminate expression of the encoded mutant FUS protein. The methods and compositions can effectively treat neurodegenerative diseases such as frontotemporal dementia (FTD) and amyotrophic lateral sclerosis (ALS). The methods and compositions involve targeting one or more single nucleotide polymorphisms (SNPs) that are non-pathogenic FUS coding variants linked to and in proximity (e.g., upstream or 5′) to FUS gene mutations correlated with the neurodegenerative conditions or diseases. The FUS gene can be heterozygous with a mutation on FUS allele. Such a mutation in one FUS allele can be a dominant mutation. The methods can edit (e.g. inactivate or remove) the mutation. In some cases, the mutant FUS allele is edited in one or more sites of FUS exons 1-4, for example in FUS exon 3 or FUS exon 4.

Compositions can be used to edit FUS genes. Such compositions can, for example, include one or more of the guide RNAs with an RNA sequence having at least 80%, at least 85%, at least 90%, or at least 93%, or at least 95%, or at least 96%, or at least 97%, or at least 98% sequence identity to any of SEQ ID NOs: 9-14. The compositions can also include a nuclease. The nuclease can be a Cas nuclease. For example, the compositions can include one or ribonucleoprotein complexes, each with one or more guide RNA and a nuclease. In some cases, expression cassettes or expression vectors can be used to provide expression of the guide RNA(s) and the nuclease(s) in the cells.

Using the methods and compositions described herein, just four different guide RNAs could treat up to 64% of FUS-FTD/ALS patients.

The FUS alleles can be edited in cells that are maintained in vitro in a culture medium, and then the modified cells can be administered to a subject to treat the neurodegenerative conditions or diseases. For example, the cells to be modified can be neuronal cells, and after modification, the modified neuronal cells can be administered to a subject. The cells to be modified can be from a subject with a neurodegenerative condition or disease, and once modified the cells can be reintroduced into the subject (i.e., the modified cells are autologous to the subject to be treated). The cells can be administered locally, for example, into neuronal cells or to neuronal tissues.

In some cases, the FUS gene can be edited in vivo by editing an endogenous gene within a subject. For example, the subject can be administered ribonucleoprotein complexes that include one or more guide RNA and one or more nucleases. In other cases, the subject can be administered expression cassettes or expression vectors to provide expression of the guide RNA(s) and the nuclease(s) in the subject's cells.

FUS presents a rare combination of three features that make it an excellent candidate for allele-specific editing. First, FUS-FTD/ALS is a dominant negative disease, caused by the presence of a single mutant FUS gene. Second, FUS is haplosufficient, so deletion of one copy of the gene has no known deleterious effect on cell or tissue health. Third, FUS contains two common SNPs early in the gene coding sequence, providing targets for CRISPR editing that are shared among a large portion of the potential patient population. For any heterozygous patient, the SNPs targeted by the guide RNAs described herein provide a specific locus for CRISPR-Cas9 knockout of the patient's mutant allele, independent of the mutation itself. The selected SNP targets occur early in the FUS coding exons, so CRISPR-induced indels at these locations result in premature termination in exon 6 (out of the 15 FUS exons) and subsequent nonsense-mediated decay of the mutant mRNA, minimizing the risk of creating a new C-terminal disrupting mutation.

The methods and compositions described herein are highly useful for performing such allele-specific editing.

### DESCRIPTION OF THE FIGURES

FIGS. 1A-1D illustrate methods and constructs for generating differentiated motor neurons, for example, that have FUS mutations. FIG. 1A is a schematic diagram of a doxycycline-inducible hNIL transgene that encodes the NGN2, ISL1, and LHX3human transcription factors used to induce differentiation. A selectable marker cassette is shown in red that is flanked by loxP sites (triangles) for removal. Homology arms (HA) are shown that target the hNIL transgene to the CLYBL safe harbor locus. FIG. 1B shows cells that were differentiated by inducing expression of the hNIL transgene shown in FIG. 1A. The cells were stained with HB9 (red, motor neuron marker), beta III-tubulin (green, neuronal marker), and nuclei marker DAPI (blue). Scale bar=50 mM. FIG. 1C shows time lapse images illustrating spheroid differentiation and rapid neurite outgrowth within hours of plating the cells. FIG. 1D shows images of differentiated spheroid fixed and stained for the neurofilament light polypeptide (NEFL; green), and DAPI (blue).

FIGS. 2A-2D illustrate FUS protein structure, FUS mutations, and the structure of the FUS genetic locus. FIG. 2A is a schematic of FUS protein structure, showing the repetitive region (QGSY-rich), RNA binding domains (RGG), DNA binding domain (RRM), zinc finger domain (ZF), and nuclear localization signal (PY). Pathogenic mutations have been reported in nearly every domain, with many putatively disrupting the C-terminal NLS. FIG. 2B shows a map of the FUS coding sequence and the locations of common SNPs in exon 3 (SNP3, rs741810) and exon 4 (SNP4, rs1052352). FIG. 2C illustrates that SNP3 and SNP4 are in linkage disequilibrium resulting in three haplotypes in humans. The frequencies of each haplotype are shown in the 1000 Genomes Project (across all populations), as well as the expected frequency of human heterozygosity for SNP3, SNP4, or both, as calculated by Hardy-Weinberg equilibrium. FIG. 2D illustrates the expected frequency of heterozygosity at SNP3, SNP4, or both, graphed by population in the 1000 Genomes project.

FIGS. 3A-3B illustrate allele-specific editing targeting coding SNPs. FIG. 3A is a schematic illustrating the spCas9 guide RNA targets on each allele of SNP3 or SNP4 in FUS within the KOLF heterozygous background. Each guide pair (i.e. g3, g4, g4*, color coded purple, orange, green) differs only by the single nucleotide of their target SNP. Multiple heterozygous cell lines with engineered mutations that were created on each allele (HapB or HapC) independently. As shown here, KOLF-R521H-HapB could be targeted by guides 3C, 4T, or 4T*, while Kolf-R521H-HapC could be targeted by guides 3A, 4C, and 4C *. FIG. 3B graphically illustrates editing efficiency and specificity of each gRNA was tested in iPSCs that were homozygous for either the Target (efficiency) or Off-Target (specificity) allele. The rate of indel creation in KOLF is expected to be half the rate of the target allele. The guide RNAs targeting SNPs 3C, 3A, and 4C were highly specific. The lead guide RNA is indicated by an asterisk. Alternate gRNA targeting of SNP4T with different Cas species (e.g., SaCas9, Cas12, Casx) can be to increase specificity.

FIGS. 4A-4B illustrate that cells under stress temporarily suspend translation of many proteins and sequester mRNA components of the translation machinery in TIA-1 positive stress granules. FIG. 4A shows that during heat shock stress (44° C. for 1 hour), TIA-1 (green) accumulates in stress granules in all control and mutant lines. FUS (red) normally localizes to the nucleus, but mislocalizes to cytoplasmic aggregates in C-terminal FUS mutants. The cell lines shown are normal control (human induced pluripotent stem KOLF2 cells), prion-domain mutation (R216C) cells, C-terminal nuclear localization signal (NLS) truncation (R495X) cells, and two C-terminal NLS point mutations (R521H, FUS-P1). Blue, DAPI. FIG. 4B shows enlarged images from FIG. 4A. Arrows highlight cytoplasmic stress granules.

FIGS. 5A-5B illustrate removal of deleterious FUS mutations from neuronal cells. FIG. 5A illustrates that dominant FUS mutations cause aggregation of FUS protein, RNA, and other proteins. The methods and compositions described herein can reverse this phenotype by specific editing of the mutant allele. FIG. 5B illustrates targeting of two coding SNPs in the FUS gene that can be targeted using the methods and compositions described herein to provide allele specific CRISPR editing and thereby selectively inactivate the mutant FUS allele.

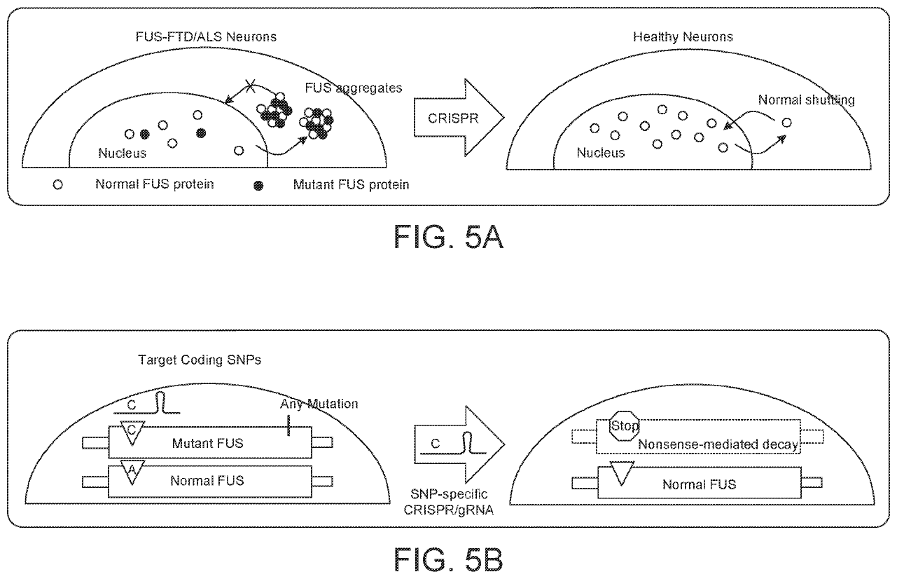

FIGS. 6A-6B depict the knockout of FUS protein by SNP-targeted CRISPR editing to visualize FUS protein in human cells.

### DETAILED DESCRIPTION

Methods and compositions are described herein that are useful to treat pathological mutation in the fused in sarcoma (FUS) gene. The FUS gene is also called ALS6, ETM4, FUS1, HNRNPP2, POMP75, TLS, and altFUS.

The compositions can include guide RNAs that can remove FUS genetic mutations, leaving one functional non-mutant FUS allele to provide the RNA-binding role and the RNA processing functions of FUS proteins. Examples of such guide RNAs (gRNAs) include those with any of SEQ ID NOs:9-14. Designing gRNAs that target the variant alleles allow for treatment and prevention of genetic diseases correlated with a pathogenic mutation for any heterozygous person.

The fused in sarcoma (FUS) gene encodes a multifunctional protein component of the heterogeneous nuclear ribonucleoprotein (hnRNP) complex. The FUS protein belongs to the FET family of RNA-binding and DNA-binding proteins which have been implicated in cellular processes that include regulation of gene expression, maintenance of genomic integrity and mRNA/microRNA processing. Alternative splicing results in multiple transcript variants.

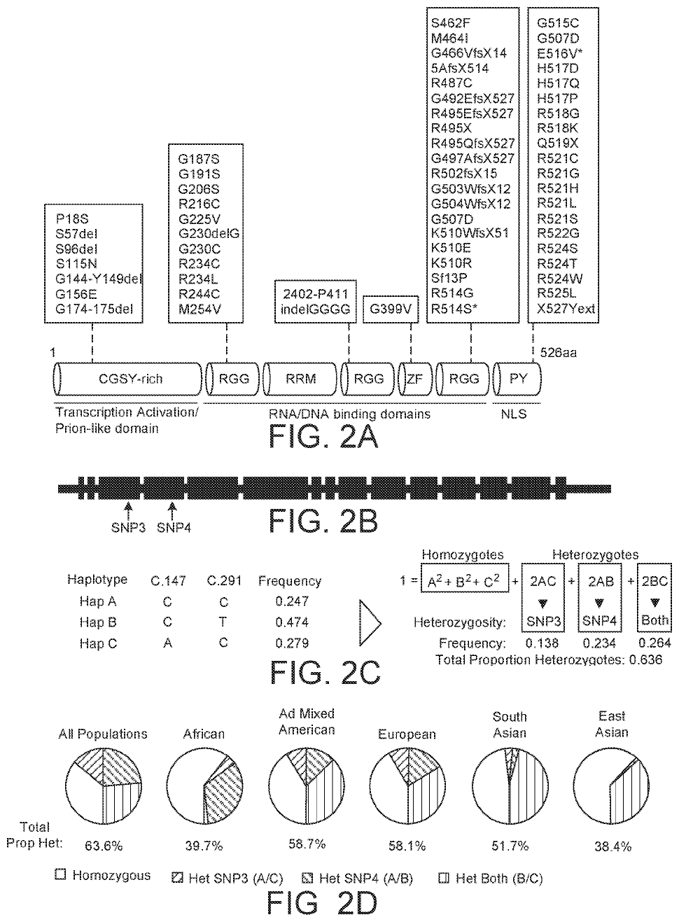

The FUS protein shuttles continuously between the nucleus and cytoplasm for mRNA export and stress granule formation (Wheeler et al. Elife 5 (2016)). Aberrant translocation of mutant FUS and its subsequent retention in RNA granules may be an underlying mechanism for FUS-ALS/FTD. Mutations causing FUS-FTD/ALS primarily occur in the N-terminal prion-like domain and the C-terminal nuclear localization signal (NLS) (FIG. 2). Mutations in the prion-like domain lead to aberrant cytoplasmic aggregations (also called pathologic RNA granules or phase transition) of FUS in vitro (Patel et al. Cell 162, 1066-1077 (2015)), while defect in nuclear import leads to cytoplasmic mislocalization and over-recruitment to granules during cellular stress (Wheeler et al. Elife 5 (2016)).

Analysis indicates that FUS may be the fourth most common cause of familial ALS in Europe, and the second most common cause of ALS in Asia. FUS mutations result in dominant negative ALS, as well as FTD (to a lesser extent).

Pathological mutations in the FUS gene are among the most frequent known genetic causes of frontotemporal dementia (FTD) and amyotrophic lateral sclerosis (ALS), collectively termed FUS-FTD/ALS (Sabatelli et al. 2013; Shang and Huang 2016; Hofmann et al. 2019). A diverse set of over 60 dominant negative mutations in FUS have been identified in human patients and lead to incurable early-onset dementia and rapid motor neuron degeneration leading to death. The FUS protein is critical for mRNA transcription, DNA damage repair, and neuronal function. However, strong genetic and experimental data indicate that FUS is haplosufficient, i.e. a single normal FUS allele could support normal neurological function (Cacheiro et al. 2019).

FUS protein is mainly localized in the nucleus, but shuttles between the nucleus and the cytosol. A hallmark of neuronal dysfunction pathology is the presence of cytoplasmic inclusions of mutated proteins in the brain and spinal cord of FUS-FTD/ALS patients. Recruitment of mutant FUS to stress granules, including cytoplasmic ribonucleoprotein granules where stalled translation complexes accumulate, indicates that disruption of stress granule function might contribute to disease pathology. Stress granules assemble rapidly through liquid-liquid phase separation in response to unexpected cellular stresses in healthy cells. However, persistent stress granule formation can lead to development of toxic aggregations of proteins with intrinsically-disordered prion-like domains, including FUS and TDP43.

FUS-FTD/ALS is inherited in a dominant negative fashion, where a single mutant allele of the FUS gene results in disease. Moreover, FUS is haplosufficient in mice, meaning that animals with just a single allele are healthy.

The FUS gene (gene ID 2521) is located on chromosome 16 at 16p11.2 (NC_000016.10; positions 31180110 . . . 31194871).

A sequence for a human FUS isoform 1 protein is available as NCBI accession no. NP_004951.1, shown below as SEQ ID NO:1.

| | |
|---|---|
| 1 | MASNDYTQQA TQSYGAYPTQ PGQGYSQQSS QPYGQQSYSG |
| 41 | YSQSTDTSGY GQSSYSSYGQ SQNTGYGTQS TPQGYGSTGG |
| 81 | YGSSQSSQSS YGQQSSYPGY GQQPAPSSTS GSYGSSSQSS |
| 121 | SYGQPQSGSY SQQPSYGGQQ QSYGQQQSYN PPQGYGQQNQ |
| 161 | YNSSSGGGGG GGGGGNYGQD QSSMSSGGGS GGGYGNQDQS |
| 201 | GGGGSGGYGQ QDRGGRGRGG SGGGGGGGGG GYNRSSGGYE |
| 241 | PRGRGGGRGG RGGMGGSDRG GENKFGGPRD QGSRHDSEQD |
| 281 | NSDNNTIFVQ GLGENVTIES VADYFKQIGI IKTNKKTGQP |
| 321 | MINLYTDRET GKLKGEATVS FDDPPSAKAA IDWEDGKEFS |
| 361 | GNPIKVSFAT RRADENRGGG NGRGGRGRGG PMGRGGYGGG |
| 401 | GSGGGGRGGF PSGGGGGGGQ QRAGDWKCPN PTCENMNESW |
| 441 | RNECNQCKAP KPDGPGGGPG GSHMGGNYGD DRRGGRGGYD |
| 481 | RGGYRGRGGD RGGFRGGRGG GDRGGFGPGK MDSRGEHRQD |
| 521 | RRERPY |

A cDNA encoding the FUS isoform 1 protein with the SEQ ID NO:1 sequence is available from the NCBI database as accession no. NM_004960 and provided below as SEQ ID NO:2.

| | |
|---|---|
| 1 | GCTCAGTCCT CCAGGCGTCG GTACTCAGCG GTGTTGGAAC |
| 41 | TTCGTTGCTT GCTTGCCTGT GCGCGCGTGC GCGGACATGG |
| 81 | CCTCAAACGA TTATACCCAA CAAGCAACCC AAAGCTATGG |
| 121 | GGCCTACCCC ACCCAGCCCG GGCAGGGCTA TTCCCAGCAG |
| 161 | AGCAGTCAGC CCTACGGACA GCAGAGTTAC AGTGGTTATA |
| 201 | GCCAGTCCAC GGACACTTCA GGCTATGGCC AGAGCAGCTA |
| 241 | TTCTTCTTAT GGCCAGAGCC AGAACACAGG CTATGGAACT |
| 281 | CAGTCAACTC CCCAGGGATA TGGCTCGACT GGCGGCTATG |
| 321 | GCAGTAGCCA GAGCTCCCAA TCGTCTTACG GGCAGCAGTC |
| 361 | CTCCTACCCT GGCTATGGCC AGCAGCCAGC TCCCAGCAGC |
| 401 | ACCTCGGGAA GTTACGGTAG CAGTTCTCAG AGCAGCAGCT |
| 441 | ATGGGCAGCC CCAGAGTGGG AGCTACAGCC AGCAGCCTAG |
| 481 | CTATGGTGGA CAGCAGCAAA GCTATGGACA GCAGCAAAGC |
| 521 | TATAATCCCC CTCAGGGCTA TGGACAGCAG AACCAGTACA |
| 561 | ACAGCAGCAG TGGTGGTGGA GGTGGAGGTG GAGGTGGAGG |
| 601 | TAACTATGGC CAAGATCAAT CCTCCATGAG TAGTGGTGGT |
| 641 | GGCAGTGGTG GCGGTTATGG CAATCAAGAC CAGAGTGGTG |
| 681 | GAGGTGGCAG CGGTGGCTAT GGACAGCAGG ACCGTGGAGG |
| 721 | CCGCGGCAGG GGTGGCAGTG GTGGCGGCGG CGGCGGCGGC |
| 761 | GGTGGTGGTT ACAACCGCAG CAGTGGTGGC TATGAACCCA |
| 801 | GAGGTCGTGG AGGTGGCCGT GGAGGCAGAG GTGGCATGGG |
| 841 | CGGAAGTGAC CGTGGTGGCT TCAATAAATT TGGTGGCCCT |
| 881 | CGGGACCAAG GATCACGICA TGACTCCGAA CAGGATAATT |
| 921 | CAGACAACAA CACCATCTTT GTGCAAGGCC TGGGTGAGAA |
| 961 | TGTTACAATT GAGTCTGTGG CTGATTACTT CAAGCAGATT |
| 1001 | GGTATTATTA AGACAAACAA GAAAACGGGA CAGCCCATGA |
| 1041 | TTAATTTGTA CACAGACAGG GAAACTGGCA AGCTGAAGGG |
| 1081 | AGAGGCAACG GTCTCTTTTG ATGACCCACC TTCAGCTAAA |
| 1121 | GCAGCTATTG ACTGGTTTGA TGGTAAAGAA TTCTCCGGAA |
| 1161 | ATCCTATCAA GGTCTCATTT GCTACTCGCC GGGCAGACTT |
| 1201 | TAATCGGGGT GGTGGCAATG GTCGTGGAGG CCGAGGGCGA |
| 1241 | GGAGGACCCA TGGGCCGTGG AGGCTATGGA GGTGGTGGCA |
| 1281 | GTGGTGGTGG TGGCCGAGGA GGATTTCCCA GTGGAGGTGG |
| 1321 | TGGCGGTGGA GGACAGCAGC GAGCTGGTGA CTGGAAGTGT |
| 1361 | CCTAATCCCA CCTGTGAGAA TATGAACTTC TCTTGGAGGA |
| 1401 | ATGAATGCAA CCAGTGTAAG GCCCCTAAAC CAGATGGCCC |
| 1441 | AGGAGGGGGA CCAGGTGGCT CTCACATGGG GGGTAACTAC |
| 1481 | GGGGATGATC GTCGTGGTGG CAGAGGAGGC TATGATCGAG |
| 1521 | GCGGCTACCG GGGCCGCGGC GGGGACCGTG GAGGCTTCCG |
| 1561 | AGGGGGCCGG GGTGGTGGGG ACAGAGGTGG CTTTGGCCCT |
| 1601 | GGCAAGATGG ATTCCAGGGG TGAGCACAGA CAGGATCGCA |
| 1641 | GGGAGAGGCC GTATTAATTA GCCTGGCTCC CCAGGTTCTG |
| 1681 | GAACAGCTTT TTGTCCTGTA CCCAGTGTTA CCCTCGTTAT |
| 1721 | TTTGTAACCT TCCAATTCCT GATCACCCAA GGGTTTTTTT |
| 1761 | GTGTCGGACT ATGTAATTGT AACTATACCT CIGGTTCCCA |
| 1801 | TTAAAAGTGA CCATTTTAGT TAAA |

A sequence for a human FUS isoform 2 protein is available as NCBI accession no. NP_001164105.1, shown below as SEQ ID NO:3.

| | |
|---|---|
| 1 | MASNDYTQQA TQSYGAYPTQ PGQGYSQQSS QPYGQQSYSG |
| 41 | YSQSTDTSGY GQSSYSSYGQ SQNSYGTQST PQGYGSTGGY |
| 81 | GSSQSSQSSY GQQSSYPGYG QQPAPSSTSG SYGSSSQSSS |
| 121 | YGQPQSGSYS QQPSYGGQQQ SYGQQQSYNP PQGYGQQNQY |
| 161 | NSSSGGGGGG GGGGNYGQDQ SSMSSGGGSG GGYGNQDQSG |
| 201 | GGGSGGYGQQ DRGGRGRGGS GGGGGGGGGG YNRSSGGYEP |
| 241 | RGRGGGRGGR GGMGGSDRGG FNKEGGPRDQ GSRHDSEQDN |
| 281 | SDNNTIFVQG LGENVTIESV ADYFKQIGII KTNKKTGQPM |
| 321 | INLYTDRETG KLKGEATVSF DDPPSAKAAI DWFDGKEFSG |
| 361 | NPIKVSFATR RADENRGGGN GRGGRGRGGP MGRGGYGGGG |
| 401 | SGGGGRGGFP SGGGGGGGQQ RAGDWKCPNP TCENMNESWR |
| 441 | NECNQCKAPK PDGPGGGPGG SHMGGNYGDD RRGGRGGYDR |
| 481 | GGYRGRGGDR GGFRGGRGGG DRGGFGPGKM DSRGEHRQDR |
| 521 | RERPY |

A cDNA encoding the FUS isoform 2 protein with the SEQ ID NO:3 sequence is available from the NCBI database as accession no. NM_001170634.1.

A sequence for a human FUS isoform 3 protein is available as NCBI accession no. NP_001164408.1, shown below as SEQ ID NO:4.

| | |
|---|---|
| 1 | MASNDYTQQA TQSYGAYPTQ PGQGYSQQSS QPYGQQSYSG |
| 41 | YSQSTDTSGY GQSSYSSYGQ SQNTGYGTQS TPQGYGSTGG |
| 81 | YGSSQSSQSS YGQQSSYPGY GQQPAPSSTS GSYGSSSQSS |
| 121 | SYGQPQSGSY SQQPSYGGQQ QSYGQQQSYN PPQGYGQQNQ |
| 161 | YNSSSGGGGG GNYGQDQSSM SSGGGSGGGY GNQDQSGGGG |
| 201 | SGGYGQQDRG GRGRGGSGGG GGGGGGGYNR SSGGYEPRGR |
| 241 | GGGRGGRGGM GGSDRGGENK FGGPRDQGSR HDSEQDNSDN |
| 281 | NTIFVQGLGE NVTIESVADY FKQIGIIKIN KKTGQPMINL |
| 321 | YTDRETGKLK GEATVSFDDP PSAKAAIDWF DGKEFSGNPI |
| 361 | KVSFATRRAD FNRGGGNGRG GRGRGGPMGR GGYGGGGSGG |
| 401 | GGRGGFPSGG GGGGGQQRAG DWKCPNPTCE NMNFSWRNEC |
| 441 | NQCKAPKPDG PGGGPGGSHM GGNYGDDRRG GRGGYDRGGY |
| 481 | RGRGGDRGGF RGGRGGGDRG GFGPGKMDSR GEHRQDRRER |
| 521 | PY |

A cDNA encoding the FUS isoform 3 protein with the SEQ ID NO:4 sequence is available from the NCBI database as accession no. NM_001170937.1.

A sequence for a human FUS isoform X3 protein is available as NCBI accession no. XP_005255290.1, shown below as SEQ ID NO:5.

| | |
|---|---|
| 1 | MAIKTRVVEV AAVAMDSRTV EAAAGVAVVA AAAAAVVVTT |
| 41 | AAVVAMNPEV VEVAVEAEVA WGPRDQGSRH DSEQDNSDNN |
| 81 | TIFVQGLGEN VTIESVADYF KQIGIIKINK KTGQPMINLY |
| 121 | TDRETGKLKG EATVSFDDPP SAKAAIDWED GKEFSGNPIK |
| 161 | VSFATRRADF NRGGGNGRGG RGRGGPMGRG GYGGGGSGGG |
| 201 | GRGGFPSGGG GGGGQQRAGD WKCPNPTCEN MNESWRNECN |
| 241 | QCKAPKPDGP GGGPGGSHMG GNYGDDRRGG RGGYDRGGYR |
| 281 | GRGGDRGGFR GGRGGGDRGG FGPGKMDSRG EHRQDRRERP |
| 321 | Y |

A cDNA encoding the FUS isoform X3 protein with the SEQ ID NO:5 sequence is available from the NCBI database as accession no. XM_005255233.5.

Another sequence for a human FUS isoform X3 protein is available as NCBI accession no. XP_011544084.1, shown below as SEQ ID NO:6.

| | |
|---|---|
| 1 | MAIKTRVVEV AAVAMDSRTV EAAAGVAVVA AAAAAVVVTT |
| 41 | AAVVAMNPEV VEVAVEAEVA WGPRDQGSRH DSEQDNSDNN |
| 81 | TIFVQGLGEN VTIESVADYF KQIGIIKINK KTGQPMINLY |
| 121 | TDRETGKLKG EATVSFDDPP SAKAAIDWED GKEFSGNPIK |
| 161 | VSFATRRADF NRGGGNGRGG RGRGGPMGRG GYGGGGSGGG |
| 201 | GRGGFPSGGG GGGGQQRAGD WKCPNPTCEN MNFSWRNECN |
| 241 | QCKAPKPDGP GGGPGGSHMG GNYGDDRRGG RGGYDRGGYR |
| 281 | GRGGDRGGFR GGRGGGDRGG FGPGKMDSRG EHRQDRRERP |
| 321 | Y |

A cDNA encoding the FUS isoform X3 protein with the SEQ ID NO:6 sequence is available from the NCBI database as accession no. XM_011545782.2.

A sequence for a human FUS isoform X1 protein is available as NCBI accession no. XP_011544083.1, shown below as SEQ ID NO:7.

```
    1 MASNDYTQQA TQSYGAYPTQ PGQGYSQQSS QPYGQQSYSG
  41 YSQSTDTSGY GQSSYSSYGQ SQNTGYGTQS TPQGYGSTGG
  81 YGSSQSSQSS YGQQSSYPGY GQQPAPSSTS GSYGSSSQSS
121 SYGQPQSGSY SQQPSYGGQQ QSYGQQQSYN PPQGYGQQNQ
161 YNSSSGGGGG GGGNYGQDQS SMSSGGGSGG GYGNQDQSGG
201 GGSGGYGQQD RGGRGRGGSG GGGGGGGGGY NRSSGGYEPR
241 GRGGGRGGRG GMGGSDRGGF NKFGGPRDQG SRHDSEQDNS
281 DNNTIFVQGL GENVTIESVA DYFKQIGIIK TNKKTGQPMI
321 NLYTDRETGK LKGEATVSFD DPPSAKAAID WEDGKEFSGN
361 PIKVSFATRR ADENRGGGNG RGGRGRGGPM GRGGYGGGGS
401 GGGGRGGFPS GGGGGGGQQR AGDWKCPNPT CENMNESWRN
441 ECNQCKAPKP DGPGGGPGGS HMGGNYGDDR RGGRGGYDRG
481 GYRGRGGDRG GFRGGRGGGD RGGFGPGKMD SRGEHRQDRR
521 ERPY
```

A cDNA encoding the FUS isoform X1 protein with the SEQ ID NO:7 sequence is available from the NCBI database as accession no. XM_011545781.1.

A sequence for a human FUS isoform X2 protein is available as NCBI accession no. XP_024305989.1, shown below as SEQ ID NO:8.

| | |
|---|---|
| 1 | MASNDYTQQA TQSYGAYPTQ PGQGYSQQSS QPYGQQSYSG |
| 41 | YSQSTDTSGY GQSSYSSYGQ SQNSYGTQST PQGYGSTGGY |
| 81 | GSSQSSQSSY GQQSSYPGYG QQPAPSSTSG SYGSSSQSSS |
| 121 | YGQPQSGSYS QQPSYGGQQQ SYGQQQSYNP PQGYGQQNQY |
| 161 | NSSSGGGGGG GGNYGQDQSS MSSGGGSGGG YGNQDQSGGG |
| 201 | GSGGYGQQDR GGRGRGGSGG GGGGGGGGYN RSSGGYEPRG |
| 241 | RGGGRGGRGG MGGSDRGGEN KFGGPRDQGS RHDSEQDNSD |
| 281 | NNTIFVQGLG ENVTIESVAD YFKQIGIIKT NKKTGQPMIN |
| 321 | LYTDRETGKL KGEATVSFDD PPSAKAAIDW FDGKEFSGNP |
| 361 | IKVSFATRRA DENRGGGNGR GGRGRGGPMG RGGYGGGGSG |
| 401 | GGGRGGFPSG GGGGGGQQRA GDWKCPNPTC ENMNESWRNE |
| 441 | CNQCKAPKPD GPGGGPGGSH MGGNYGDDRR GGRGGYDRGG |
| 481 | YRGRGGDRGG FRGGRGGGDR GGFGPGKMDS RGEHRQDRRE |
| 521 | RPY |

A cDNA encoding the FUS isoform X2 protein with the SEQ ID NO:8 sequence is available from the NCBI database as accession no. XM_024450221.1.

Sequence variations and mutations can naturally occur. For example, isoforms, variants, and mutants of the proteins and nucleic acids described herein can be detected and modified using the methods and compositions described herein. The isoforms, variants, and mutants can, for example, have sequences with between 55-100% sequence identity to a reference FUS sequence, for example with at least 55% sequence identity, at least 60%, at least 70%, at least 80%, at least 90%, at least 95%, at least 96%, at least 97%, at least 98%, or at least 99% sequence identity to any of the sequences described herein.

### Therapeutic Methods

A therapeutic method is described herein involving deleting the mutant allele while preserving the normal allele to promote development and maintenance of healthy neural tissue.

In the field of gene editing, it is commonly proposed to use a mutation as its own target for CRISPR-Cas9 based knockout of a mutant allele. However, this approach is untenable with FUS-related diseases and conditions for two reasons. First, over 60 different mutations have been identified in patients with FUS-FTD/ALS (FIG. 2A). Not only would the time and cost required to develop, optimize, and undergo FDA testing of individual guide RNAs (gRNAs) for each mutation be prohibitive, but most of these mutations are not directly targetable by a CRISPR-Cas9 gRNA in the first place. Second, regarding disease manifestation, a majority of FUS mutations occur in the C-terminus of the protein and result in disruption of the nuclear localization signal present here (FIG. 2A). As a result, any editing strategy that targets these mutations and introduces an insertion or deletion (indel) in the end of the gene risks creating a new, and potentially worse disease-causing mutation.

As mentioned, over 60 different FUS mutations cause neurodegenerative diseases such as FUS-FTD/ALS via a dominant negative mechanism. Since only one allele of FUS is required for normal function, eliminating disease alleles is curative. A key challenge of current technologies is the diversity of FUS disease mutations, making it difficult (or impossible) to target each disease allele.

However, the compositions and methods described herein target two common SNPs that occur early in the FUS gene, such that targeting either of these SNPs results in inactivation of one allele. The selected SNPs are very common in the human population. Estimates indicate that more than 65% of FUS-FTD/ALS patients could be treated with just four gRNAs that target each potential heterozygous allele.

Sequences of six different gRNAs are shown in Table 1 that target each of the SNP alleles.

TABLE 1
Guide RNAs for Targeting and Inactivating SNPs

| | gRNA | | | | SEQ ID |
|---|---|---|---|---|---|
| Name | Pair | SNP | Allele | gRNA Sequence | NO: |
| SNP3-C | g3 | rs741810 | 147C | uccacggacacuucaggcua | 9 |
| SNP3-A | g3 | rs741810 | 147A | uccacggacacuucaggaua | 10 |
| SNP4-C | g4 | rs1052352 | 291C | gggcagcaguccuccuaccc | 11 |
| SNP4-T | g4 | rs1052352 | 291T | gggcagcaguccuccuaucc | 12 |
| SNP4-C* | g4* | rs1052352 | 291C | cugcuggccauagccagggu | 13 |
| SNP4-T* | g4* | rs1052352 | 291T | cugcuggccauagccaggau | 14 |

These six gRNAs shown in Table 1 target two SNPs that appear in exon 3 (rs741810, c.147C/A) and exon 4 (rs1052352, c.291C/T) of FUS (FIG. 2A-2B, 3A). By definition, SNPs are common, non-pathogenic variations in the human genome that occur commonly in the population. The rs741810 and rs1052352 SNPs are clinically benign (see NCBI SNP database at website ncbi.nlm.nih.gov/snp), but the rs741810 and rs1052352 SNPs are linked to downstream FUS mutations that are correlated with neurodegenerative conditions or diseases.

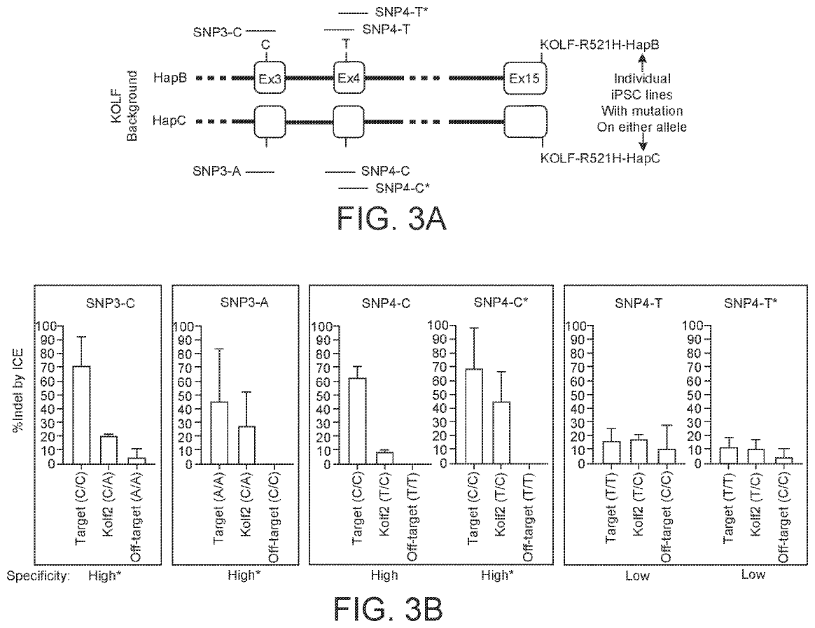

In the case of the rs741810 and rs1052352 SNPs, they are inherited together in one of three potential pairings, or haplotypes, that result in 65% of patients being heterozygous at one or both locations (FIG. 2C-2D). Thus, for any heterozygous patient, these SNPs provide a specific target for CRISPR-Cas9 knockout of their mutant allele, independent of the mutation itself (FIG. 3A-3B). Importantly, these SNPs occur early in the gene, so cleavage and/or mutations (indels) at these locations result in premature termination in exon 6 (out of 15) and subsequent nonsense-mediated decay of the mutant mRNA, minimizing the risk of creating a new C-terminal disrupting mutation.

Only a few gRNAs are therefore needed to effectively treat 65% or more of FUS-FTD/ALS patients.

### Gene Editing Technology

Described herein are allele-specific guide RNAs that can repair, knockdown or knockout the expression of an undesired polypeptide encoded by a variant (mutant) FUS allele. The CRISPR-Cas9 genome-editing system can be used to delete/correct FUS mutations that are correlated with cardiomyopathy. For example, one or two guide RNAs (gRNAs) can be used to recognize one or more target sequence in a subject's genome, and a corrective nuclease can act as a pair of scissors to cleave a single-strand or a double-strand of genomic DNA. Mutations in the genome that are near the cleavage site(s) can be repaired by an endogenous Non-Homologous End Joining (NHEJ) or Homology Directed Repair (HDR) repair pathway. Hence, the guide RNAs guide the corrective nuclease to cleave the targeted genomic site for deletion and/or repair by endogenous mechanisms. Examples of the specific guide RNA sequences provided herein are shown in Table 1.

The allele-specific guide RNAs can cut the variant alleles in the same position within exon 3 or 4 of the FUS allele. The guide RNAs are also allele-specific and can make allele-specific excision/deletion of at least 2,500 base pairs of the FUS genetic locus. Such deletion can remove problematic FUS mutations and reduce the incidence and/or severity of phenotypes associated with these mutations.

The Cas system can recognize any sequence in the genome that matches 20 bases of a gRNA. However, each gRNA must also be adjacent to a “Protospacer Adjacent Motif” (PAM), which is invariant for each type of Cas protein, because the PAM binds directly to the Cas protein. See Doudna et al., Science 346 (6213): 1077, 1258096 (2014); and Jinek et al., Science 337:816-21 (2012). Hence, the guide RNAs have a PAM site sequence that can be bound by a Cas protein.

When the Cas system was first described for Cas9, with a “NGG” PAM site, the PAM was somewhat limiting in that it required a GG in the right orientation to the site to be targeted. Different Cas9 species have now been described with different PAM sites. See Jinek et al., Science 337:816-21 (2012); Ran et al., Nature 520:186-91 (2015); and Zetsche et al., Cell 163:759-71 (2015). In addition, mutations in the PAM recognition domain (Table 2) have increased the diversity of PAM sites for SpCas9 and SaCas9. See Kleinstiver et al., Nat Biotechnol 33:1293-1298 (2015); and Kleinstiver et al., Nature 523:481-5 (2015).

Table 2 summarizes information about PAM sites.

TABLE 2
PAM sites

| | PAM sites |
|---|---|
| SpCas9 | NGG |
| SpCas9 VRER variant | NGCG |
| SpCas9 EQR variant | NGAG |
| SpCas9 VQR variant | NGAN or NGNG |
| SaCas9 | NNGRRT |
| SaCas9, KKH variant | NNNRRT |
| FnCas2 (Cpf1) | TTN |

DNA annotations:
N = A, C, T or G
R = Purine, A or G

Note that the guide RNAs for SpCas9 and SaCas9 cover 20 bases in the 5′direction of the PAM site, while for FnCas2 (Cpf1) the guide RNA covers 20 bases to 3′ of the PAM.

There are a number of different types of corrective nucleases and systems that can be used for gene editing. The corrective nuclease employed can in some cases be any DNA binding protein with corrective nuclease activity. Examples of corrective nuclease include Streptococcus pyogenes Cas (SpCas9) nucleases, Staphylococcus aureus Cas9 (SpCas9) nucleases, Francisella novicida Cas2 (FnCas2, also called dFnCpf1) nucleases, Zinc Finger Nucleases (ZFN), Meganuclease, Transcription activator-like effector nucleases (TALEN), Fok-I nucleases, any DNA binding protein with nuclease activity, any DNA binding protein bound to a corrective nuclease, or any combinations thereof. However, the CRISPR-Cas systems are generally the most widely used. In some cases, the corrective nuclease is therefore a Cas nuclease.

CRISPR-Cas systems are generally divided into two classes. The class 1 system contains types I, III and IV, and the class 2 system contains types II, V, and VI. The class 1 CRISPR-Cas system uses a complex of several Cas proteins, whereas the class 2 system only uses a single Cas protein with multiple domains. The class 2 CRISPR-Cas system is usually preferable for gene-engineering applications because of its simplicity and ease of use.

A variety of Cas nucleases can be employed in the methods described herein. Three species that have been best characterized are provided as examples. The most commonly used Cas nuclease is a Streptococcus pyogenes Cas9, (SpCas9). More recently described forms of Cas include Staphylococcus aureus Cas9 (SaCas9) and Francisella novicida Cas2 (FnCas2, also called FnCpf1). Jinek et al., Science 337:816-21 (2012); Qi et al., Cell 152:1173-83 (2013); Ran et al., Nature 520:186-91 (2015); Zetsche et al., Cell 163:759-71 (2015).

One example of an amino acid sequence for Streptococcus pyogenes Cas9 (SpCas9) nuclease is provided below (SEQ ID NO:15).

| | |
|---|---|
| 1 | MDKKYSIGLD IGTNSVGWAV ITDEYKVPSK KFKVLGNTDR |
| 41 | HSIKKNLIGA LLFDSGETAE ATRLKRTARR RYTRRKNRIC |
| 81 | YLQEIFSNEM AKVDDSFFHR LEESFLVEED KKHERHPIFG |
| 121 | NIVDEVAYHE KYPTIYHLRK KLVDSTDKAD LRLIYLALAH |
| 161 | MIKFRGHFLI EGDLNPDNSD VDKLFIQLVQ TYNQLFEENP |
| 201 | INASGVDAKA ILSARLSKSR RLENLIAQLP GEKKNGLFGN |
| 241 | LIALSLGLTP NFKSNEDLAE DAKLQLSKDT YDDDLDNLLA |
| 281 | QIGDQYADLF LAAKNLSDAI LLSDILRVNT EITKAPLSAS |
| 321 | MIKRYDEHHQ DLTLLKALVR QQLPEKYKEI FFDQSKNGYA |
| 361 | GYIDGGASQE EFYKFIKPIL EKMDGTEELL VKLNREDLLR |
| 401 | KQRTFDNGSI PHQIHLGELH AILRRQEDFY PFLKDNREKI |
| 441 | EKILTFRIPY YVGPLARGNS RFAWMTRKSE ETITPWNFEE |
| 481 | VVDKGASAQS FIERMINEDK NLPNEKVLPK HSLLYEYFTV |
| 521 | YNELTKVKYV TEGMRKPAFL SGEQKKAIVD LLFKTNRKVT |
| 561 | VKQLKEDYFK KIECFDSVEI SGVEDRENAS LGTYHDLLKI |
| 601 | IKDKDELDNE ENEDILEDIV LTLTLFEDRE MIEERLKTYA |
| 641 | HLFDDKVMKQ LKRRRYTGWG RLSRKLINGI RDKQSGKTIL |
| 681 | DFLKSDGFAN RNEMQLIHDD SLTFKEDIQK AQVSGQGDSL |
| 721 | HEHIANLAGS PAIKKGILQT VKVVDELVKV MGRHKPENIV |
| 761 | IEMARENQTT QKGQKNSRER MKRIEEGIKE LGSQILKEHP |
| 801 | VENTQLQNEK LYLYYLQNGR DMYVDQELDI NRLSDYDVDH |
| 841 | IVPQSFLKDD SIDNKVLTRS DKNRGKSDNV PSEEVVKKMK |
| 881 | NYWRQLLNAK LITQRKFDNL TKAERGGLSE LDKAGFIKRQ |
| 921 | LVETRQITKH VAQILDSRMN TKYDENDKLI REVKVITLKS |
| 961 | KLVSDFRKDF QFYKVREINN YHHAHDAYLN AVVGTALIKK |
| 1001 | YPKLESEFVY GDYKVYDVRK MIAKSEQEIG KATAKYFFYS |
| 1041 | NIMNFFKTEI TLANGEIRKR PLIETNGETG EIVWDKGRDF |
| 1081 | ATVRKVLSMP QVNIVKKTEV QTGGFSKESI LPKRNSDKLI |
| 1121 | ARKKDWDPKK YGGFDSPTVA YSVLVVAKVE KGKSKKLKSV |
| 1161 | KELLGITIME RSSFEKNPID FLEAKGYKEV KKDLIIKLPK |
| 1201 | YSLFELENGR KRMLASAGEL QKGNELALPS KYVNFLYLAS |
| 1241 | HYEKLKGSPE DNEQKQLFVE QHKHYLDEII EQISEFSKRV |
| 1281 | ILADANLDKV LSAYNKHRDK PIREQAENII HLFTLINLGA |
| 1321 | PAAFKYFDTT IDRKRYTSTK EVLDATLIHQ SITGLYETRI |
| 1361 | DLSQLGGD |

A cDNA that encodes the Streptococcus pyogenes Cas9 (SpCas9) is provided below (SEQ ID NO:16).

| | |
|---|---|
| 1 | GACAAGAAGT ACAGCATCGG CCTGGACATC GGCACCAACT |
| 41 | CTGTGGGCTG GGCCGTGATC ACCGACGAGT ACAAGGTGCC |
| 81 | CAGCAAGAAA TTCAAGGTGC TGGGCAACAC CGACCGGCAC |
| 121 | AGCATCAAGA AGAACCTGAT CGGAGCCCTG CTGTTCGACA |
| 161 | GCGGCGAAAC AGCCGAGGCC ACCCGGCTGA AGAGAACCGC |
| 201 | CAGAAGAAGA TACACCAGAC GGAAGAACCG GATCTGCTAT |
| 241 | CTGCAAGAGA TCTTCAGCAA CGAGATGGCC AAGGTGGACG |
| 281 | ACAGCTTCTT CCACAGACTG GAAGAGTCCT TCCTGGTGGA |
| 321 | AGAGGATAAG AAGCACGAGC GGCACCCCAT CTTCGGCAAC |
| 361 | ATCGTGGACG AGGTGGCCTA CCACGAGAAG TACCCCACCA |
| 401 | TCTACCACCT GAGAAAGAAA CTGGTGGACA GCACCGACAA |
| 441 | GGCCGACCTG CGGCTGATCT ATCTGGCCCT GGCCCACATG |
| 481 | ATCAAGTTCC GGGGCCACTT CCTGATCGAG GGCGACCTGA |
| 521 | ACCCCGACAA CAGCGACGTG GACAAGCTGT TCATCCAGCT |
| 561 | GGTGCAGACC TACAACCAGC TGTTCGAGGA AAACCCCATC |
| 601 | AACGCCAGCG GCGTGGACGC CAAGGCCATC CTGTCTGCCA |
| 641 | GACTGAGCAA GAGCAGACGG CTGGAAAATC TGATCGCCCA |
| 681 | GCTGCCCGGC GAGAAGAAGA ATGGCCTGTT CGGAAACCTG |
| 721 | ATTGCCCTGA GCCTGGGCCT GACCCCCAAC TTCAAGAGCA |
| 761 | ACTTCGACCT GGCCGAGGAT GCCAAACTGC AGCTGAGCAA |
| 801 | GGACACCTAC GACGACGACC TGGACAACCT GCTGGCCCAG |
| 841 | ATCGGCGACC AGTACGCCGA CCTGTTTCTG GCCGCCAAGA |
| 881 | ACCTGTCCGA CGCCATCCTG CTGAGCGACA TCCTGAGAGT |
| 921 | GAACACCGAG ATCACCAAGG CCCCCCTGAG CGCCTCTATG |
| 961 | ATCAAGAGAT ACGACGAGCA CCACCAGGAC CTGACCCTGC |
| 1001 | TGAAAGCTCT CGTGCGGCAG CAGCTGCCTG AGAAGTACAA |
| 1041 | AGAGATTTTC TTCGACCAGA GCAAGAACGG CTACGCCGGC |
| 1081 | TACATTGACG GCGGAGCCAG CCAGGAAGAG TTCTACAAGT |
| 1121 | TCATCAAGCC CATCCTGGAA AAGATGGACG GCACCGAGGA |
| 1161 | ACTGCTCGTG AAGCTGAACA GAGAGGACCT GCTGCGGAAG |
| 1201 | CAGCGGACCT TCGACAACGG CAGCATCCCC CACCAGATCC |
| 1241 | ACCTGGGAGA GCTGCACGCC ATTCTGCGGC GGCAGGAAGA |
| 1281 | TTTTTACCCA TTCCTGAAGG ACAACCGGGA AAAGATCGAG |
| 1321 | AAGATCCTGA CCTTCCGCAT CCCCTACTAC GTGGGCCCTC |
| 1361 | TGGCCAGGGG AAACAGCAGA TTCGCCTGGA TGACCAGAAA |
| 1401 | GAGCGAGGAA ACCATCACCC CCTGGAACTT CGAGGAAGTG |
| 1441 | GTGGACAAGG GCGCTTCCGC CCAGAGCTTC ATCGAGCGGA |
| 1481 | TGACCAACTT CGATAAGAAC CTGCCCAACG AGAAGGTGCT |
| 1521 | GCCCAAGCAC AGCCTGCTGT ACGAGTACTT CACCGTGTAT |
| 1561 | AACGAGCTGA CCAAAGTGAA ATACGTGACC GAGGGAATGA |
| 1601 | GAAAGCCCGC CTTCCTGAGC GGCGAGCAGA AAAAGGCCAT |
| 1641 | CGTGGACCTG CTGTTCAAGA CCAACCGGAA AGTGACCGTG |
| 1681 | AAGCAGCTGA AAGAGGACTA CTTCAAGAAA ATCGAGTGCT |
| 1721 | TCGACTCCGT GGAAATCTCC GGCGTGGAAG ATCGGTTCAA |
| 1761 | CGCCTCCCTG GGCACATACC ACGATCTGCT GAAAATTATC |
| 1801 | AAGGACAAGG ACTTCCTGGA CAATGAGGAA AACGAGGACA |
| 1841 | TTCTGGAAGA TATCGTGCTG ACCCTGACAC TGTTTGAGGA |
| 1881 | CAGAGAGATG ATCGAGGAAC GGCTGAAAAC CTATGCCCAC |
| 1921 | CTGTTCGACG ACAAAGTGAT GAAGCAGCTG AAGCGGCGGA |
| 1961 | GATACACCGG CTGGGGCAGG CTGAGCCGGA AGCTGATCAA |
| 2001 | CGGCATCCGG GACAAGCAGT CCGGCAAGAC AATCCTGGAT |
| 2041 | TTCCTGAAGT CCGACGGCTT CGCCAACAGA AACTTCATGC |
| 2081 | AGCTGATCCA CGACGACAGC CTGACCTTTA AAGAGGACAT |
| 2121 | CCAGAAAGCC CAGGTGTCCG GCCAGGGCGA TAGCCTGCAC |
| 2161 | GAGCACATTG CCAATCTGGC CGGCAGCCCC GCCATTAAGA |
| 2201 | AGGGCATCCT GCAGACAGTG AAGGTGGTGG ACGAGCTCGT |
| 2241 | GAAAGTGATG GGCCGGCACA AGCCCGAGAA CATCGTGATC |
| 2281 | GAAATGGCCA GAGAGAACCA GACCACCCAG AAGGGACAGA |
| 2321 | AGAACAGCCG CGAGAGAATG AAGCGGATCG AAGAGGGCAT |
| 2361 | CAAAGAGCTG GGCAGCCAGA TCCTGAAAGA ACACCCCGTG |
| 2401 | GAAAACACCC AGCTGCAGAA CGAGAAGCTG TACCTGTACT |
| 2441 | ACCTGCAGAA TGGGCGGGAT ATGTACGTGG ACCAGGAACT |
| 2481 | GGACATCAAC CGGCTGTCCG ACTACGATGT GGACCATATC |
| 2521 | GTGCCTCAGA GCTTTCTGAA GGACGACTCC ATCGACAACA |
| 2561 | AGGTGCTGAC CAGAAGCGAC AAGAACCGGG GCAAGAGCGA |
| 2601 | CAACGTGCCC TCCGAAGAGG TCGTGAAGAA GATGAAGAAC |
| 2641 | TACTGGCGGC AGCTGCTGAA CGCCAAGCTG ATTACCCAGA |
| 2681 | GAAAGTTCGA CAATCTGACC AAGGCCGAGA GAGGCGGCCT |
| 2721 | GAGCGAACTG GATAAGGCCG GCTTCATCAA GAGACAGCTG |
| 2761 | GTGGAAACCC GGCAGATCAC AAAGCACGTG GCACAGATCC |
| 2801 | TGGACTCCCG GATGAACACT AAGTACGACG AGAATGACAA |
| 2841 | GCTGATCCGG GAAGTGAAAG TGATCACCCT GAAGTCCAAG |
| 2881 | CTGGTGTCCG ATTTCCGGAA GGATTTCCAG TTTTACAAAG |
| 2921 | TGCGCGAGAT CAACAACTAC CACCACGCCC ACGACGCCTA |
| 2961 | CCTGAACGCC GTCGTGGGAA CCGCCCTGAT CAAAAAGTAC |
| 3001 | CCTAAGCTGG AAAGCGAGTT CGTGTACGGC GACTACAAGG |
| 3041 | TGTACGACGT GCGGAAGATG ATCGCCAAGA GCGAGCAGGA |
| 3081 | AATCGGCAAG GCTACCGCCA AGTACTTCTT CTACAGCAAC |
| 3121 | ATCATGAACT TTTTCAAGAC CGAGATTACC CTGGCCAACG |
| 3161 | GCGAGATCCG GAAGCGGCCT CTGATCGAGA CAAACGGCGA |
| 3201 | AACCGGGGAG ATCGTGTGGG ATAAGGGCCG GGATTTTGCC |
| 3241 | ACCGTGCGGA AAGTGCTGAG CATGCCCCAA GTGAATATCG |
| 3281 | TGAAAAAGAC CGAGGTGCAG ACAGGCGGCT TCAGCAAAGA |
| 3321 | GTCTATCCTG CCCAAGAGGA ACAGCGATAA GCTGATCGCC |
| 3361 | AGAAAGAAGG ACTGGGACCC TAAGAAGTAC GGCGGCTTCG |
| 3401 | ACAGCCCCAC CGTGGCCTAT TCTGTGCTGG TGGTGGCCAA |
| 3441 | AGTGGAAAAG GGCAAGTCCA AGAAACTGAA GAGTGTGAAA |
| 3481 | GAGCTGCTGG GGATCACCAT CATGGAAAGA AGCAGCTTCG |
| 3521 | AGAAGAATCC CATCGACTTT CTGGAAGCCA AGGGCTACAA |
| 3561 | AGAAGTGAAA AAGGACCTGA TCATCAAGCT GCCTAAGTAC |
| 3601 | TCCCTGTTCG AGCTGGAAAA CGGCCGGAAG AGAATGCTGG |
| 3641 | CCTCTGCCGG CGAACTGCAG AAGGGAAACG AACTGGCCCT |
| 3681 | GCCCTCCAAA TATGTGAACT TCCTGTACCT GGCCAGCCAC |
| 3721 | TATGAGAAGC TGAAGGGCTC CCCCGAGGAT AATGAGCAGA |
| 3761 | AACAGCTGTT TGTGGAACAG CACAAGCACT ACCTGGACGA |
| 3801 | GATCATCGAG CAGATCAGCG AGTTCTCCAA GAGAGTGATC |
| 3841 | CTGGCCGACG CTAATCTGGA CAAAGTGCTG TCCGCCTACA |
| 3881 | ACAAGCACCG GGATAAGCCC ATCAGAGAGC AGGCCGAGAA |
| 3921 | TATCATCCAC CTGTTTACCC TGACCAATCT GGGAGCCCCT |
| 3961 | GCCGCCTTCA AGTACTTTGA CACCACCATC GACCGGAAGA |
| 4001 | GGTACACCAG CACCAAAGAG GTGCTGGACG CCACCCTGAT |
| 4041 | CCACCAGAGC ATCACCGGCC TGTACGAGAC ACGGATCGAC |
| 4081 | CTGTCTCAGC TGGGAGGCGA C |

An amino acid sequence for a Francisella novicida Cas2 (FnCas2, also called FnCpf1) is shown below (SEQ ID NO:17).

| | |
|---|---|
| 1 | MTQFEGFTNL YQVSKTLRFE LIPQGKTLKH IQEQGFIEED |
| 41 | KARNDHYKEL KPIIDRIYKT YADQCLQLVQ LDWENLSAAI |
| 81 | DSYRKEKTEE TRNALIEEQA TYRNAIHDYF IGRTDNLTDA |
| 121 | INKRHAEIYK GLFKAELENG KVLKQLGTVT TTEHENALLR |
| 161 | SFDKFTTYFS GFYENRKNVF SAEDISTAIP HRIVQDNFPK |
| 201 | FKENCHIFTR LITAVPSLRE HFENVKKAIG IFVSTSIEEV |
| 241 | FSFPFYNQLL TQTQIDLYNQ LLGGISREAG TEKIKGLNEV |
| 281 | LNLAIQKNDE TAHIIASLPH RFIPLFKQIL SDRNTLSFIL |
| 321 | EEFKSDEEVI QSFCKYKTLL RNENVLETAE ALFNELNSID |
| 361 | LTHIFISHKK LETISSALCD HWDTLRNALY ERRISELTGK |
| 401 | ITKSAKEKVQ RSLKHEDINL QEIISAAGKE LSEAFKQKTS |
| 441 | EILSHAHAAL DQPLPTTLKK QEEKEILKSQ LDSLLGLYHL |
| 481 | LDWFAVDESN EVDPEFSARL TGIKLEMEPS LSFYNKARNY |
| 521 | ATKKPYSVEK FKLNFQMPTL ASGWDVNKEK NNGAILFVKN |
| 561 | GLYYLGIMPK QKGRYKALSF EPTEKTSEGF DKMYYDYFPD |
| 601 | AAKMIPKCST QLKAVTAHFQ THTTPILLSN NFIEPLEITK |
| 641 | EIYDLNNPEK EPKKFQTAYA KKTGDQKGYR EALCKWIDFT |
| 681 | RDFLSKYTKT TSIDLSSLRP SSQYKDLGEY YAELNPLLYH |
| 721 | ISFQRIAEKE IMDAVETGKL YLFQIYNKDF AKGHHGKPNL |
| 761 | HTLYWTGLFS PENLAKTSIK LNGQAELFYR PKSRMKRMAH |
| 801 | RLGEKMLNKK LKDQKTPIPD TLYQELYDYV NHRLSHDLSD |
| 841 | EARALLPNVI TKEVSHEIIK DRRFTSDKFF FHVPITLNYQ |
| 881 | AANSPSKENQ RVNAYLKEHP ETPIIGIDRG ERNLIYITVI |
| 921 | DSTGKILEQR SLNTIQQFDY QKKLDNREKE RVAARQAWSV |
| 961 | VGTIKDLKQG YLSQVIHEIV DLMIHYQAVV VLENLNFGFK |
| 1001 | SKRIGIAEKA VYQQFEKMLI DKLNCLVLKD YPAEKVGGVL |
| 1041 | NPYQLTDQFT SFAKMGTQSG FLFYVPAPYT SKIDPLTGFV |
| 1081 | DPFVWKTIKN HESRKHFLEG FDFLHYDVKT GDFILHFKMN |
| 1121 | RNLSFQRGLP GEMPAWDIVE EKNETQFDAK GTPFIAGKRI |
| 1161 | VPVIENHRFT GRYRDLYPAN ELIALLEEKG IVERDGSNIL |
| 1201 | PKLLENDDSH AIDTMVALIR SVLQMRNSNA ATGEDYINSP |
| 1241 | VRDLNGVCFD SRFQNPEWPM DADANGAYHI ALKGQLLLNH |
| 1281 | LKESKDLKLQ NGISNQDWLA YIQELRN |

A cDNA that encodes the foregoing Francisella novicida Cas2 (FnCas2, also called dFnCpf1) polypeptide is shown below (SEQ ID NO:18).

| 1 | ATGACACAGT TCGAGGGCTT TACCAACCTG TATCAGGTGA |
|---|---|
| 41 | GCAAGACACT GCGGTTTGAG CTGATCCCAC AGGGCAAGAC |
| 81 | CCTGAAGCAC ATCCAGGAGC AGGGCTTCAT CGAGGAGGAC |
| 121 | AAGGCCCGCA ATGATCACTA CAAGGAGCTG AAGCCCATCA |
| 161 | TCGATCGGAT CTACAAGACC TATGCCGACC AGTGCCTGCA |
| 201 | GCTGGTGCAG CTGGATTGGG AGAACCTGAG CGCCGCCATC |
| 241 | GACTCCTATA GAAAGGAGAA AACCGAGGAG ACAAGGAACG |
| 281 | CCCTGATCGA GGAGCAGGCC ACATATCGCA ATGCCATCCA |
| 321 | CGACTACTTC ATCGGCCGGA CAGACAACCT GACCGATGCC |
| 361 | ATCAATAAGA GACACGCCGA GATCTACAAG GGCCTGTTCA |
| 401 | AGGCCGAGCT GTTTAATGGC AAGGTGCTGA AGCAGCTGGG |
| 441 | CACCGTGACC ACAACCGAGC ACGAGAACGC CCTGCTGCGG |
| 481 | AGCTTCGACA AGTTTACAAC CTACTTCTCC GGCTTTTATG |
| 521 | AGAACAGGAA GAACGTGTTC AGCGCCGAGG ATATCAGCAC |
| 561 | AGCCATCCCA CACCGCATCG TGCAGGACAA CTTCCCCAAG |
| 601 | TTTAAGGAGA ATTGTCACAT CTTCACACGC CTGATCACCG |
| 721 | CCGTGCCCAG CCTGCGGGAG CACTTTGAGA ACGTGAAGAA |
| 761 | GGCCATCGGC ATCTTCGTGA GCACCTCCAT CGAGGAGGTG |
| 801 | TTTTCCTTCC CTTTTTATAA CCAGCTGCTG ACACAGACCC |
| 841 | AGATCGACCT GTATAACCAG CTGCTGGGAG GAATCTCTCG |
| 881 | GGAGGCAGGC ACCGAGAAGA TCAAGGGCCT GAACGAGGTG |
| 921 | CTGAATCTGG CCATCCAGAA GAATGATGAG ACAGCCCACA |
| 961 | TCATCGCCTC CCTGCCACAC AGATTCATCC CCCTGTTTAA |
| 1001 | GCAGATCCTG TCCGATAGGA ACACCCTGTC TTTCATCCTG |
| 1041 | GAGGAGTTTA AGAGCGACGA GGAAGTGATC CAGTCCTTCT |
| 1081 | GCAAGTACAA GACACTGCTG AGAAACGAGA ACGTGCTGGA |
| 1121 | GACAGCCGAG GCCCTGTTTA ACGAGCTGAA CAGCATCGAC |
| 1161 | CTGACACACA TCTTCATCAG CCACAAGAAG CTGGAGACAA |
| 1201 | TCAGCAGCGC CCTGTGCGAC CACTGGGATA CACTGAGGAA |
| 1241 | TGCCCTGTAT GAGCGGAGAA TCTCCGAGCT GACAGGCAAG |
| 1281 | ATCACCAAGT CTGCCAAGGA GAAGGTGCAG CGCAGCCTGA |
| 1321 | AGCACGAGGA TATCAACCTG CAGGAGATCA TCTCTGCCGC |
| 1361 | AGGCAAGGAG CTGAGCGAGG CCTTCAAGCA GAAAACCAGC |
| 1401 | GAGATCCTGT CCCACGCACA CGCCGCCCTG GATCAGCCAC |
| 1441 | TGCCTACAAC CCTGAAGAAG CAGGAGGAGA AGGAGATCCT |
| 1481 | GAAGTCTCAG CTGGACAGCC TGCTGGGCCT GTACCACCTG |
| 1521 | CTGGACTGGT TTGCCGTGGA TGAGTCCAAC GAGGTGGACC |
| 1561 | CCGAGTTCTC TGCCCGGCTG ACCGGCATCA AGCTGGAGAT |
| 1601 | GGAGCCTTCT CTGAGCTTCT ACAACAAGGC CAGAAATTAT |
| 1641 | GCCACCAAGA AGCCCTACTC CGTGGAGAAG TTCAAGCTGA |
| 1681 | ACTTTCAGAT GCCTACACTG GCCTCTGGCT GGGACGTGAA |
| 1721 | TAAGGAGAAG AACAATGGCG CCATCCTGTT TGTGAAGAAC |
| 1761 | GGCCTGTACT ATCTGGGCAT CATGCCAAAG CAGAAGGGCA |
| 1801 | GGTATAAGGC CCTGAGCTTC GAGCCCACAG AGAAAACCAG |
| 1841 | CGAGGGCTTT GATAAGATGT ACTATGACTA CTTCCCTGAT |
| 1881 | GCCGCCAAGA TGATCCCAAA GTGCAGCACC CAGCTGAAGG |
| 1921 | CCGTGACAGC CCACTTTCAG ACCCACACAA CCCCCATCCT |
| 1961 | GCTGTCCAAC AATTTCATCG AGCCTCTGGA GATCACAAAG |
| 2001 | GAGATCTACG ACCTGAACAA TCCTGAGAAG GAGCCAAAGA |
| 2041 | AGTTTCAGAC AGCCTACGCC AAGAAAACCG GCGACCAGAA |
| 2081 | GGGCTACAGA GAGGCCCTGT GCAAGTGGAT CGACTTCACA |
| 2121 | AGGGATTTTC TGTCCAAGTA TACCAAGACA ACCTCTATCG |
| 2161 | ATCTGTCTAG CCTGCGGCCA TCCTCTCAGT ATAAGGACCT |
| 2201 | GGGCGAGTAC TATGCCGAGC TGAATCCCCT GCTGTACCAC |
| 2241 | ATCAGCTTCC AGAGAATCGC CGAGAAGGAG ATCATGGATG |
| 2281 | CCGTGGAGAC AGGCAAGCTG TACCTGTTCC AGATCTATAA |
| 2321 | CAAGGACTTT GCCAAGGGCC ACCACGGCAA GCCTAATCTG |
| 2361 | CACACACTGT ATTGGACCGG CCTGTTTTCT CCAGAGAACC |
| 2401 | TGGCCAAGAC AAGCATCAAG CTGAATGGCC AGGCCGAGCT |
| 2441 | GTTCTACCGC CCTAAGTCCA GGATGAAGAG GATGGCACAC |
| 2481 | CGGCTGGGAG AGAAGATGCT GAACAAGAAG CTGAAGGATC |
| 2521 | AGAAAACCCC AATCCCCGAC ACCCTGTACC AGGAGCTGTA |
| 2561 | CGACTATGTG AATCACAGAC TGTCCCACGA CCTGTCTGAT |
| 2601 | GAGGCCAGGG CCCTGCTGCC CAACGTGATC ACCAAGGAGG |
| 2641 | TGTCTCACGA GATCATCAAG GATAGGCGCT TTACCAGCGA |
| 2681 | CAAGTTCTTT TTCCACGTGC CTATCACACT GAACTATCAG |
| 2721 | GCCGCCAATT CCCCATCTAA GTTCAACCAG AGGGTGAATG |
| 2761 | CCTACCTGAA GGAGCACCCC GAGACACCIA TCATCGGCAT |
| 2801 | CGATCGGGGC GAGAGAAACC TGATCTATAT CACAGTGATC |
| 2841 | GCCTCCACCG GCAAGATCCT GGAGCAGCGG AGCCTGAACA |
| 2881 | CCATCCAGCA GTTTGATTAC CAGAAGAAGC TGGACAACAG |
| 2921 | GGAGAAGGAG AGGGTGGCAG CAAGGCAGGC CTGGTCTGTG |
| 2961 | GTGGGCACAA TCAAGGATCT GAAGCAGGGC TATCTGAGCC |
| 3001 | AGGTCATCCA CGAGATCGTG GACCTGATGA TCCACTACCA |
| 3041 | GGCCGTGGTG GTGCTGGAGA ACCTGAATTT CGGCTTTAAG |
| 3081 | AGCAAGAGGA CCGGCATCGC CGCGAAGGCC GTGTACCAGC |
| 3121 | AGTTCGAGAA GATGCTGATC GATAAGCTGA ATTGCCTGGT |
| 3161 | GCTGAAGGAC TATCCAGCAG AGAAAGTGGG AGGCGTGCTG |
| 3201 | AACCCATACC AGCTGACAGA CCAGTTCACC TCCTTTGCCA |
| 3241 | AGATGGGCAC CCAGTCTGGC TTCCTGTTTT ACGTGCCTGC |
| 3281 | CCCATATACA TCTAAGATCG ATCCCCTGAC CGGCTTCGTG |
| 3321 | GACCCCTTCG TGTGGAAAAC CATCAAGAAT CACGAGAGCC |
| 3361 | GCAAGCACTT CCTGGAGGGC TTCGACTTTC TGCACTACGA |
| 3401 | CGTGAAAACC GGCGACTTCA TCCTGCACTT TAAGATGAAC |
| 3441 | AGAAATCTGT CCTTCCAGAG GGGCCTGCCC GGCTTTATGC |
| 3481 | CTGCATGGGA TATCGTGTTC GAGAAGAACG AGACACAGTT |
| 3521 | TGACGCCAAG GGCACCCCTT TCATCGCCGG CAAGAGAATC |
| 3561 | GTGCCAGTGA TCGAGAATCA CAGATTCACC GGCAGATACC |
| 3601 | GGGACCTGTA TCCTGCCAAC GAGCTGATCG CCCTGCTGGA |
| 3641 | GGAGAAGGGC ATCGTGTTCA GGGATGGCTC CAACATCCTG |
| 3681 | CCAAAGCTGC TGGAGAATGA CGATTCTCAC GCCATCGACA |
| 3721 | CCATGGTGGC CCTGATCCGC AGCGTGCTGC AGATGCGGAA |
| 3761 | CTCCAATGCC GCCACAGGCG AGGACTATAT CAACAGCCCC |
| 3801 | GTGCGCGATC TGAATGGCGT GTGCTTCGAC TCCCGGTTTC |
| 3841 | AGAACCCAGA GTGGCCCATG GACGCCGATG CCAATGGCGC |
| 3881 | CTACCACATC GCCCTGAAGG GCCAGCTGCT GCTGAATCAC |
| 3921 | CTGAAGGAGA GCAAGGATCT GAAGCTGCAG AACGGCATCT |
| 3961 | CCAATCAGGA CIGGCTGGCC TACATCCAGG AGCTGCGCAA |
| 4001 | C |

### Guide RNA Delivery

There are different ways to deliver guide RNAs and corrective nucleases. The first and probably the most straightforward approach is to use a vector-based CRISPR-Cas9 system encoding the corrective nuclease and guide RNA (e.g., sgRNA) from the same vector, thus avoiding multiple transfections of different components. The second is to deliver the mixture of the Cas9 mRNA and the sgRNA, and the third strategy is to deliver the mixture of the Cas9 protein and the sgRNA.

In some cases, the guide RNAs can be delivered to cells or administered to subjects in the form of an expression cassette or vector that can express one or more of the guide RNAs. Corrective nucleases can also be delivered to cells or administered to the subjects in the form of an expression cassette or vector that can express one or more corrective nucleases. The corrective nucleases can also be combined with their respective gRNAs and delivered as RNA-protein complexes (RNPs). Hence, the RNPs can be pre-assembled outside of the cell and introduced into the cell.

Hence, the guide RNAs can be recombinantly expressed in the cells. The corrective nuclease can also be expressed in the same cell with one or more gRNAs. The guide RNAs and corrective nucleases can be introduced in form of a nucleic acid molecules encoding the guide RNAs and/or corrective nucleases. The nucleic acid molecules encoding the guide RNAs and/or corrective nuclease proteins can be provided in expression cassettes or expression vectors.

The expression cassettes can be within vectors. Vectors can, for example, be expression vectors such as viruses or other vectors that is readily taken up by the cells. Examples of vectors that can be used include, for example, adeno-associated virus (AAV) gene transfer vectors, lentiviral vectors, retroviral vectors, herpes virus vectors, e.g., cytomegalovirus vectors, herpes simplex virus vectors, varicella zoster virus vectors, adenovirus vectors, e.g., helper-dependent adenovirus vectors, adenovirus-AAV hybrids, rabies virus vectors, vesicular stomatitis virus (VSV) vectors, coronavirus vectors, poxvirus vectors and the like. Non-viral vectors may be employed to deliver the expression vectors, e.g., liposomes, nanoparticles, microparticles, lipoplexes, polyplexes, nanotubes, and the like. In one embodiment, two or more expression vectors are administered, for instance, each encoding a distinct guide RNA, a distinct corrective nuclease, or a combination thereof.

The expression cassettes or expression vectors include promoter sequences that are operably linked to the nucleic acid segment encoding the guide RNAs, corrective nucleases, or combinations thereof. Methods for ensuring expression of a functional guide RNA, corrective nuclease or combinations thereof can involve expression from a transgene, expression cassette, or expression vector. For example, the nucleic acid segments encoding the selected guide RNAs, or combinations thereof can be present in a vector, such as for example a plasmid, cosmid, virus, bacteriophage or another vector available for genetic engineering. The coding sequences inserted in the vector can be synthesized by standard methods or isolated from natural sources. The coding sequences may further be ligated to transcriptional regulatory elements, termination sequences, and/or to other amino acid encoding sequences. Such regulatory sequences can provide initiation of transcription, internal ribosomal entry sites (IRES) (Owens, Proc. Natl. Acad. Sci. USA 98:1471-1476 (2001)) and optionally regulatory elements ensuring termination of transcription and stabilization of the transcript. Non-limiting examples for regulatory elements ensuring the initiation of transcription comprise a translation initiation codon, transcriptional enhancers such as e.g. the SV40-enhancer, insulators and/or promoters. The promoter can be a constitutive promoter, and inducible promoter, or a tissue-specific promoter. Examples of promoters that can be used include the cytomegalovirus (CMV) promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the lacUV5 promoter, the Autographa californica multiple nuclear polyhedrosis virus (AcMNPV) polyhedral promoter, or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing. Moreover, elements such as origin of replication, drug resistance gene or regulators (as part of an inducible promoter) may also be included.

The expression cassettes and/or expression vectors can be introduced into cells. The cells can be any mammalian or avian cell. For example, the cells can be human cells, or cells from a domesticated animal, a zoo animal, or an experimental animal. The cells can be obtained from a subject in need of treatment. The cells can be autologous or allogenic cells relative to a subject. In some cases, the cells can be stem cells, induced pluripotent stem cells, neuronal progenitor cells, cardiomyocytes and/or neuronal cells. The allogenic cells can be typed to match those of a subject.

The guide RNAs can also be introduced into cells or administered to subjects in the form of RNA-protein complexes (RNPs). The corrective nuclease can be pre-bound with their respective gRNAs prior to introduction into cells. The advantage RNP delivery of Cas-gRNA complexes is that complex formation it is readily controlled ex vivo and the selected Cas polypeptides can independently be complexed with selected guide RNA sequences so that the structure and compositions of the desired complexes is known with certainty. These RNPs are quite stable, with no apparent exchange of gRNAs. Hence, the nuclease-gRNA RNP can carry a selected gRNA to the site of genomic editing.

For example, Cas RNP can be prepared by incubating the Cas proteins with the selected gRNA using a molar excess of gRNA relative to protein (e.g., using about a 1:1.1 to 1:1.4 protein to gRNA molar ratio). The buffer to be used during such incubation can include 20 mM HEPES (pH 7 5), 150 mM KCl, 1 mM MgCl, 10% glycerol and 1 mM TCEP. Incubation can be done at 37° C. for about 5 minutes to about 30 minutes (usually 10 minutes is sufficient). When reference DNA or an HDR template is used, it can be added to the Cas RNP.

Nucleofection can be employed to introduce the Cas RNP into cells. See Lin et al., Enhanced homology-directed human genome engineering by controlled timing of CRISPR/Cas9 delivery. Elife 3:e04766. For example, nucleofection reactions can involve mixing approximately 1×10, ₜₒ 1×10, cells in about 10 μl to 40 μl of nucleofection reagent with about 5 p to 30 μl of RNP: DNA. In some instances, about 2×10 cells are mixed with about 20 μl of nucleofection reagent and about 10 μl RNP:DNA. After electroporation, growth media is added, and the cells are transferred to tissue culture plates for growth and evaluation. The nucleofection reagents and machines are available from Lonza (Allendale, NJ)

Thus, the invention provides compounds for use in medical therapy, such as gene therapy vectors that inhibit or prevent neurodegeneration.

### Cell Therapies

In some cases, cells can be modified in vitro and then administered to a subject. For example, cells can be contacted and/or treated with any of the guide RNAs, ribonucleoprotein complexes, expression cassettes/expression vectors or modifying agents described herein. Such modification can reduce FUS expression from a FUS allele that expresses a FUS protein correlated with a neurodegenerative condition or disease. The cells can be autologous or allogeneic to the subject so administered. For example, the cells can be obtained from a subject, then these cells can be contacted and/or treated with any of the guide RNAs, ribonucleoprotein complexes, expression cassettes/expression vectors or modifying agents described herein to generate modified cells. The cells to be modified can, for example, be epithelial cells, neuronal cells, or neural cells.

The modified cells can be expanded in culture to form a population of modified cells and the population of cells can be administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a device or a vehicle so that a population of liposomes, exosomes or microvesicles.

Cells are administered to patients at various time points, for example, as immunotherapy, or to retard or inhibit tumor growth. Administration of cells should improve the neurodegenerative condition of the subject. Treatment may comprise the cells administered alone or with any of the guide RNAs, ribonucleoprotein complexes, expression cassettes/expression vectors or modifying agents described herein. Such agents can be administered separately from or with the modified cells. For example, the modified cells may be administered prior to, during, or after administering any of the guide RNAs, ribonucleoprotein complexes, expression cassettes/expression vectors or modifying agents described herein.

### Administration

Guide RNAs, expression cassettes/expression vectors that can express the guide RNA, modified cells, or combinations thereof can be administered to subjects. Cells that have been modified to eliminate problematic FUS mutations can be administered to subjects with or without the guide RNAs, ribonucleoprotein complexes, expression cassettes/expression vectors or modifying agents described herein. Such guide RNAs, expression cassettes, expression vectors, and cells generated as described herein can be employed for tissue reconstitution or regeneration in a human patient or other subjects. Patients or subjects can be in need of such treatment. In some cases, the patients or subjects may not yet exhibit any symptoms of disease or a medical condition. However, a patient or subject to be treated may have at least one FUS allele correlated with development or existence of a neurodegeneration condition such as frontotemporal dementia (FTD) or amyotrophic lateral sclerosis (ALS), collectively termed FUS-FTD/ALS.

The guide RNAs, expression cassettes, expression vectors, and cells are administered in a manner that permits them to be incorporated into, graft or migrate to a specific tissue site, such as into neurons or neuronal tissues. Such guide RNAs, expression cassettes, expression vectors, and cells can reconstitute or regenerate functionally deficient areas of tissues, including neurons and neuronal tissues. Devices are available that can be adapted for administering cells, for example, to neurons and neuronal tissues.

For therapy, guide RNAs, expression cassettes, expression vectors, and/or neuronal cells can be administered locally or systemically. Administration can be by injection, catheter, implantable device, or the like. The guide RNAs, expression cassettes, expression vectors, and cells can be administered in any physiologically acceptable excipient or carrier that does not adversely affect the subject. For example, the guide RNAs, expression cassettes, expression vectors, and cells can be administered intravenously or through an intracranial or spinal route. Methods of administering the guide RNAs, expression cassettes, expression vectors, and/or cells to subjects, particularly human subjects, include injection or implantation of the guide RNAs, expression cassettes, expression vectors, and cells into target sites or they can be inserted into a delivery device which facilitates introduction, uptake, incorporation, or implantation of the expression cassettes, expression vectors, and cells. Such delivery devices include tubes, e.g., catheters, for introducing cells, expression vectors, and fluids into the body of a recipient subject. The tubes can additionally include a needle, e.g., a syringe, through which the cells of the invention can be introduced into the subject at a desired location. Multiple injections may be made using this procedure.

As used herein, the term “solution” includes a carrier or diluent in which the guide RNAs, expression cassettes, expression vectors, and cells of the invention remain viable and/or functional. Carriers and diluents that can be used include saline, aqueous buffer solutions, solvents and/or dispersion media. The use of such carriers and diluents are available in the art. The solution is can be sterile and fluid to the extent that easy syringeability exists.

The guide RNAs, expression cassettes, expression vectors, and cells can also be embedded in a support matrix. Suitable ingredients include matrix proteins that support or promote the incorporation of adhesion of the guide RNAs, expression cassettes, expression vectors, and modified cells. In another embodiment, the composition may include physiologically acceptable matrix scaffolds. Such physiologically acceptable matrix scaffolds can be resorbable and/or biodegradable.

In some cases, neuronal cells can be modified to express the guide RNAs and optionally the corrective nuclease. In addition, neuronal cells can be modified by the guide RNAs and corrective nucleases to generate a population of modified cells that do not have a pathological mutation in their FUS gene.

A population of modified cells generated by the methods described herein can include low percentages of non-neuronal cells (e.g., fibroblasts and/or endothelial cells). For example, a population of modified cells for use in compositions and for administration to subjects can have less than about 90% non-neuronal cells, less than about 85% non-neuronal cells, less than about 80% non-neuronal cells, less than about 75% non-neuronal cells, less than about 70% non-neuronal cells, less than about 65% non-neuronal cells, less than about 60% non-neuronal cells, less than about 55% non-neuronal cells, less than about 50% non-neuronal cells, less than about 45% non-neuronal cells, less than about 40% non-neuronal cells, less than about 35% non-neuronal cells, less than about 30% non-neuronal cells, less than about 25% non-neuronal cells, less than about 20% non-neuronal cells, less than about 15% non-neuronal cells, less than about 12% non-neuronal cells, less than about 10% non-neuronal cells, less than about 8% non-neuronal cells, less than about 6% non-neuronal cells, less than about 5% non-neuronal cells, less than about 4% non-neuronal cells, less than about 3% non-neuronal cells, less than about 2% non-neuronal cells, or less than about 1% non-neuronal cells of the total cells in the cell population.

Many cell types are capable of migrating to an appropriate site for regeneration and differentiation within a subject. To determine the suitability of various therapeutic administration regimens and dosages of cell compositions, the cells can first be tested in a suitable animal model. At one level, cells are assessed for their ability to survive and maintain their phenotype in vivo. Cells can also be assessed to ascertain whether they migrate to diseased or injured sites in vivo, or to determine an appropriate number, or dosage, of cells to be administered. Cell compositions can be administered to immunodeficient animals (such as nude mice, or animals rendered immunodeficient chemically or by irradiation). Tissues can be harvested after a period of regrowth and assessed as to whether the administered cells or progeny thereof are still present, are alive, and/or have migrated to desired or undesired locations.

Injected cells can be traced by a variety of methods. For example, cells containing or expressing a detectable label (such as green fluorescent protein, or beta-galactosidase) can readily be detected. The cells can be pre-labeled, for example, with BrdU or [³H]-thymidine, or by introduction of an expression cassette that can express green fluorescent protein, or beta-galactosidase. Alternatively, the modified cells can be detected by their expression of a cell marker that is not expressed by the animal employed for testing (for example, a human-specific antigen when injecting cells into an experimental animal). The presence and phenotype of the administered population of modified cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a neuronal phenotype.

Modified cells can be included in the compositions in varying amounts depending upon the extent of disease or the condition of the subject. For example, the compositions can be prepared in liquid form for local or systemic administration containing about 10³ to about 10¹² modified cells, or about 10⁴ to about 10¹⁰ modified cells, or about 10⁵ to about 10⁸ modified cells.

One or more RNPs containing a guide RNA or expression vectors that can express one or more guide RNAs, corrective nuclease, or a combination thereof can also be administered with or without the cells.

The guide RNA, corrective nuclease, and/or RNP with or without additional cells may be administered in a composition as a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compositions of the invention may be as a single dose, or essentially continuous over a preselected period of time, or it may be in a series of spaced doses. Both local and systemic administration is contemplated.

It will be appreciated that the amounts of guide RNAs, corrective nucleases, RNPs, and/or cells for use in treatment will vary not only with the particular carrier selected but also with the route of administration, the nature of the condition being treated and the age and condition of the patient. Ultimately, the attendant health care provider may determine proper dosage.

### Definitions

The term “about” as used herein when referring to a measurable value such as an amount, a length, and the like, is meant to encompass variations of ±20% or ±10%, including ±5%, including ±1%, and including ±0.1% from the specified value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

“Recombinant” as used herein to describe a nucleic acid molecule means a polynucleotide of genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “cell” refers to any type of cell. In some cases the cell is a neuronal cell. The cell can be in an organism or it can be maintained outside of an organism. The cell can be within a living organism and be in its normal (native) state. The term “cell” includes an individual cell or a group or population of cells. The cell(s) can be a prokaryotic, eukaryotic, or archaeon cell(s), such as a bacterial, archaeal, fungal, protist, plant, or animal cell(s). The cell(s) can be from or be within tissues, organs, and biopsies. The cell(s) can be a recombinant cell(s), a cell(s) from a cell line cultured in vitro. The cell(s) can include cellular fragments, cell components, or organelles comprising nucleic acids. In some cases, the cell(s) are human cells. The term cell(s) also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transfection, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected RNA or a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, including at least 8 to 10 amino acids, including at least 15 to 20 amino acids from a polypeptide encoded by the nucleic acid sequence. The RNA sequence or a portion thereof contains a nucleotide sequence of at least 3 to 5 nucleotides, including at least 8 to 10 nucleotides, and including at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Purified polynucleotide” refers to a polynucleotide of interest or fragment thereof which is essentially free, e.g., contains less than about 50%, including less than about 70%, and including less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, including 80%-85%, including 90-95% of the sample. Techniques for purifying polynucleotides and polypeptides of interest are well-known in the art and include, for example, ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

The term define “transduction” refers to the introduction of foreign nucleic acid to a cell through a replication-incompetent viral vector.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Mammalian cell” refers to any cell derived from a mammalian subject suitable for transfection with an engineered vector system comprising an expression system described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, including at least about 8 nucleotides, including at least about 10-12 nucleotides, and including at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks) or minimal Cas target DNA or RNA binding activity. A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR adaption system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In one embodiment, the protospacer is a defined synthetic DNA. In some embodiments, the defined synthetic DNA is at least 3, 5,10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

The following Examples describe some of the materials and experiments used in the develop of the invention.

### Example 1: Materials and Methods

This Example illustrates some of the materials and methods used in the development of the invention.

### Cell Lines

A series of isogenic induced pluripotent stem cells (iPSCs) were made that contain three different pathological FUS mutations and that express a transgenic system for rapidly producing motor-neurons (iPSC-neurons). The engineered pathological FUS mutations were a prion-like domain mutation (R216C), a C-terminal truncation (R495*), and a nuclear localization mutation (R521H).

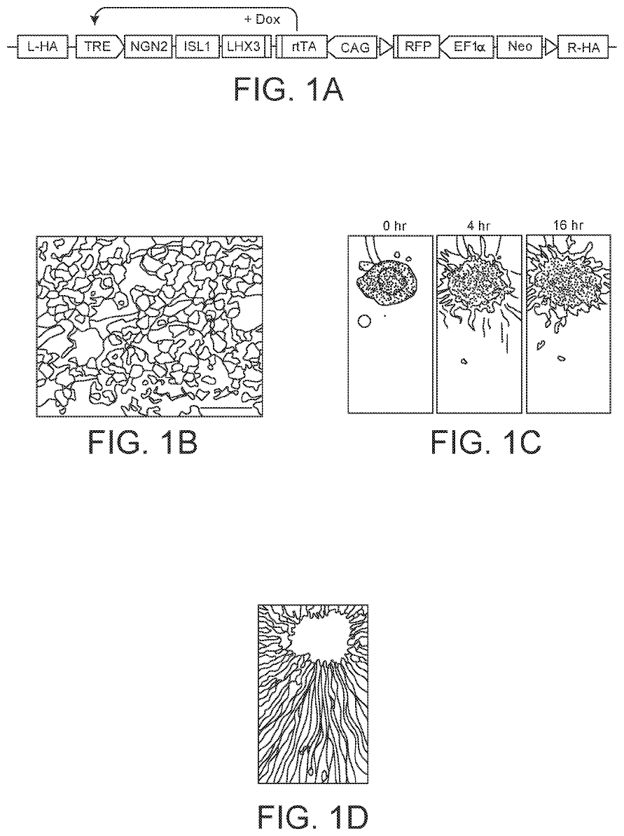

Human motor neurons were derived from an isogenic series of iPSCs that harbor the three mutations using a rapid and scalable method for the production of human motor neurons. The method involved use of a doxycycline-inducible expression cassette encoding three human transcription factors (NGN2, ISL1, and LHX3, where the combination is abbreviated “hNIL”) (FIG. 1A). The protocol can be performed in bulk or in 96-well plate format.

As shown in FIG. 1B this method efficiently generates HB9-positive motor neurons of high purity in one week. Modification of the protocol provided neuro-spheroids that formed radially projecting axons (FIG. 1C-1D). Imaging and measurement of axonal function was readily performed, particularly when the axons elongated in a consistent and predictable outward radial orientation.

Induced PSC lines with the inducible hNIL transgene integrated at the CLYBL safe-harbor locus are being generated from each of the lines listed in Table 3, where the FUS genotype of the cell line is shown as wild type (WT), mutant, or tagged with a fluorescent marker (+GFP, +Halo). The domain referred to in Table 3 indicates where the mutation or tag occurs. The background in Table 3 indicates the genetic background of each line. All lines named “KOLF” are isogenic, with mutations engineered into KOLF2.1 parental iPSCs, the control line used by iNDI (Ramos et al. Neuron 109:1080-1083 (2021). The terms iPSC, Eng, and hNIL indicate the current status of iPSC generation (iPSC), mutation or tag engineering (Eng.), and stable transduction of the hNIL motor neuron construct (hNIL), respectively (where C=complete; and P=in process).

TABLE 3
Cell Lines Obtained/Generated

| Line | Genotype | Domain | Background | iPSC | Eng. | hNIL |
|---|---|---|---|---|---|---|
| WTB | WT | n/a | Healthy, | C | n/a | C |
| | | | female control | | | |
| WTC | WT | n/a | Healthy, | C | n/a | C |
| | | | male control | | | |
| KOLF | WT | n/a | Healthy, | C | n/a | C |
| | | | male control | | | |
| WTC-FUS- | +GFP | C-Term | WTC | C | C | P |
| GFP | | | | | | |
| KOLF-FUS- | +GFP | C-Term | KOLF | C | P | P |
| GFP | | | | | | |
| KOLF-FUS- | +Halo | C-Term | KOLF | C | C | P |
| Halo | | | | | | |
| KOLF-R216C- | R216C | Prion-like | KOLF | C | C | C |
| HapB | on HapB | | | | | |
| KOLF-R216C- | R216C | Prion-like | KOLF | C | C | P |
| HapC | on HapC | | | | | |
| KOLF-R495X- | R495-stop | NLS | KOLF | C | C | C |
| HapB | on HapB | Truncation | | | | |
| KOLF-R495X- | R495-stop | NLS | KOLF | C | C | P |
| HapC | on HapC | Truncation | | | | |
| KOLF-R521H- | R521H | NLS | KOLF | C | C | C |
| HapB | on HapB | | | | | |
| KOLF-R521H- | R521H | NLS | KOLF | C | C | P |
| HapC | on HapC | | | | | |
| FUS-P1 | R521G | NLS | Patient line | C | n/a | C |
| FUS-P2 | pending | pending | Patient line | P | n/a | P |

Table 3 therefore summarizes the panel of human iPSC lines that were generated, which included wild-type controls from multiple backgrounds, an isogenic series of disease-relevant FUS mutations engineered into the KOLF background, two independent patient lines harboring FUS mutations, and two lines where FUS has been tagged at the C-terminus with GFP or Halo fluorescence tags. The KOLF parental line was a significant contribution to this panel because it contains FUS Haplotypes B and C, and thus is heterozygous for both common coding SNPs present in FUS (FIG. 3A). Subsequently, for each engineered FUS mutation, two independent lines were isolated where the mutation is present on either the B or C haplotype. This isogenic series facilitates validation of the hypothesis that targeted editing of each potential SNP allele will lead to the resolution of various FUS mutant phenotypes.

### Genetic Site Identification

The inventors have developed software (AlleleAnalyzer; Keough et al. Genome Biol. 20:167 (2019)) to identify heterozygous SNPs, across an entire genomic locus (e.g., more than 200 kb), that can be targeted by CRISPR. The inventors have also developed a new method called digital droplet excision Reporter (ddXR; Watry et al. Sci. Rep. 10:14896 (2020)) that can rapidly and precisely identify and quantify large DNA excisions (0.1 kb to >170 kb). Such tools can be used inactivate disease alleles. In addition, in pursuit of in vivo therapeutics, the inventors have developed DISCOVER-seq (Wienert et al. Science 364:286-289 (2019)), which is a method to identify and minimize off-target editing in iPSCs and in animal models.

### Example 2: Allele-Specific Genetic Modification of FUS

Heterozygous FUS mice have no apparent phenotype, and some reports indicate that there are no major problems with knocking down FUS in humans (leading to approximately a 60% knockdown). Hence, FUS is haplo-sufficient.

However, there are splicing variants of the FUS transcript. The inventors identified two common coding SNPs, in exon 3 (rs741810, c.147C/A) and exon 4 (rs1052352, c.291C/T) of FUS. The patterns of FUS these two SNPs was examined, and the inventors identified that these two SNPs could result in single guide allele-specific edits in over 50% of patients.

The targeted SNPs in FUS were each a single base variation, and the pairs of guides (g3, g4, g4*) that targeted each SNP differed by only a single nucleotide. Nevertheless, the inventors found, when editing in multiple other genes, that a single nucleotide difference was sufficient to confer high specificity to the gRNAs for their intended targets.

FIG. 2C illustrates that SNP3 and SNP4 are in linkage disequilibrium resulting in three haplotypes in humans. The frequencies of each haplotype are shown in the 1000 Genomes Project (across all populations), as well as the expected frequency of human heterozygosity for SNP3, SNP4, or both, as calculated by Hardy-Weinberg equilibrium. FIG. 2D illustrates the expected frequency of heterozygosity at SNP3, SNP4, or both, graphed by population in the 1000 Genomes project.

Six different gRNAs were designed, with the sequences shown in Table 1 to target the SNP that appears in exon 3 (rs741810, c. 147C/A) or in exon 4 (rs1052352, c.291C/T) of FUS (FIG. 2B, 3A). The KOLF-R521H-HapB could therefore be targeted by guides 3C, 4T, or 4T*, while Kolf-R521H-HapC could thus be targeted by guides 3A, 4C, and 4C* (FIG. 3A).

TABLE 1
Guide RNAs for Targeting and Inactivating SNPs

| | gRNA | | | | SEQ ID |
|---|---|---|---|---|---|
| Name | Pair | SNP | Allele | gRNA Sequence | NO: |
| SNP3-C | g3 | rs741810 | 147C | uccacggacacuucaggcua | 9 |
| SNP3-A | g3 | rs741810 | 147A | uccacggacacuucaggaua | 10 |
| SNP4-C | g4 | rs1052352 | 291C | gggcagcaguccuccuaccc | 11 |
| SNP4-T | g4 | rs1052352 | 291T | gggcagcaguccuccuaucc | 12 |
| SNP4-C* | g4* | rs1052352 | 291C | cugcuggccauagccagggu | 13 |
| SNP4-T* | g4* | rs1052352 | 291T | cugcuggccauagccaggau | 14 |

The cutting efficiency and specificity of each guide RNA with the sequences shown in Table 1 was tested in iPSCs that were homozygous for either the Target allele (allowing target modification efficiency to be detected) or Off-Target allele (allowing the specificity of modification to be detected). The KOLF2 cells used are heterozygous for these SNPs so the rate of indel creation was expected to be half the rate of the Target allele. Inference of CRISPR Edits (ICE) was used to assess the guide RNA cutting (insertion-deletion, Indel) efficiency.

Each of the six spCas9-gRNA combinations were transfected into control cell lines that are either homozygous or heterozygous for the target allele, or homozygous for the non-target allele (FIG. 3). PCR amplification and Sanger sequencing of the target site, followed by Inference of CRISPR Edits (ICE) analysis showed that the gRNA targeted the SNP3-C, SNP3-A, and SNP4-C alleles with high editing efficiencies and specificities, but only poor editing occurred at the SNP4-T allele (FIG. 3B).

FIG. 3A-3B therefore illustrate allele-specific editing of SNPs.

### Example 3: Mutant FUS Mislocalizes to Stress Granules During Cell Stress

Although FUS-FTD/ALS is a neurodegenerative disease, FUS protein is widely expressed in many cell types and tissues. Such wide-spread expression allowed evaluation of FUS mutations in different cell lines.

The expression of FUS was evaluated in several cell lines maintained under normal conditions and under heat-stress conditions. The cell lines evaluated were: normal control KOLF2 cells, KOLF2 cells with FUS R216C, FUS R495X; and FUS R521H mutations engineered into Kolf2 background, and a patient cell line (FUS-P1) with an R521G mutation. As a control, nucleolysin TIA-1 isoform expression was observed with the FUS expression. The nucleolysin TIA-1 isoform is involved in alternative pre-RNA splicing and regulation of mRNA translation by binding to AU-rich elements located in mRNA 3′ untranslated regions (3′ UTRs).

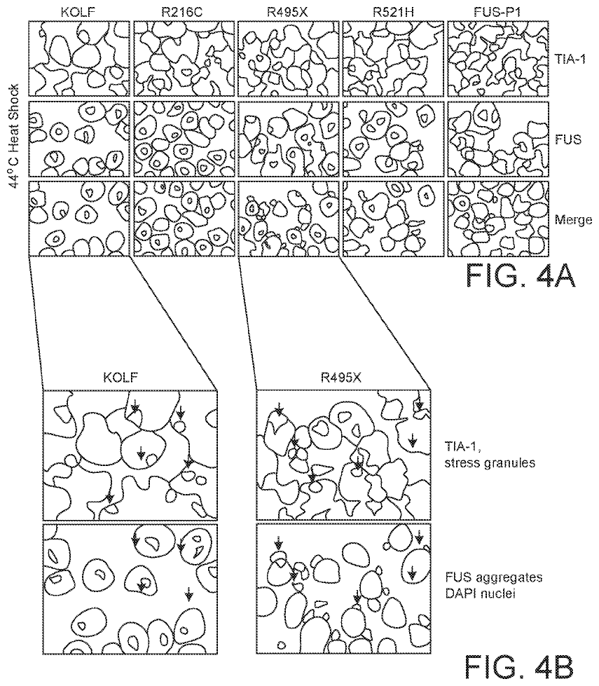

Under normal conditions (37° C.), TIA-1 (green) is present throughout the cytoplasm and nucleus, while FUS (red) localizes to the nucleus. However, as shown FIG. 4A, during heat shock stress (44° C. for 1 hour), TIA-1 (green) accumulates in stress granules in all control and mutant lines. FUS (red) normally localizes to the nucleus, but mislocalizes to cytoplasmic aggregates in C-terminal FUS mutants. FIG. 4B shows enlarged images from FIG. 1A, where the arrows highlight cytoplasmic stress granules. Hence, mutant FUS mislocalizes to stress granules during cell stress.

After a 1-hour heat shock at 44° C., both control and mutant iPSCs display cytoplasmic stress granules marked by the granule-associated RNA-binding protein TIA-1 (green) (FIG. 4B). Thus, during stress, cells temporarily suspend translation of many proteins, and sequester mRNA components of the translation machinery in cytoplasmic TIA-1 positive stress granules.

However, only lines with a mutation affecting the NLS (R495X, R521H, FUS-P1) exhibited mis-localization of FUS (red) to the stress granules, while in both the control line (KOLF) and the cell line with a prion-domain mutation (R216C), FUS remained solely nuclear (FIG. 4B). Thus, as shown in FIG. 4B, during heat shock stress (44° C.), TIA-1+ stress granules appear in all lines, but only C-terminal FUS mutants display cytoplasmic mislocalization of FUS.

Hence, for the NLS and truncation mutations, increased FUS localization to the stress granules occurs under cellular stress, resulting in a decrease of the nuclear/cytoplasmic FUS ratio. Compared to actively dividing iPSCs, terminally differentiated motor neurons cannot clear protein aggregates as efficiently. As a result, although we did not observe aggregation of FUS in the prion-domain mutation in iPSCs, such FUS aggregation and changes in stress granule dynamics may occur in motor neurons.

### Example 4: Knockout of FUS Protein by SNP-Targeted CRISPR Editing

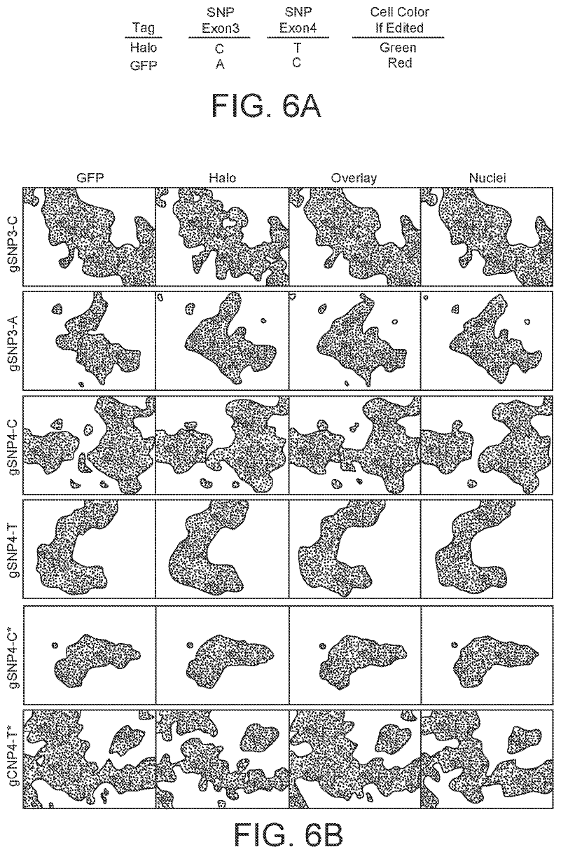

In order to visualize FUS protein in human cells, DNA sequences for green fluorescent protein (GFP) and HaloTag (“Halo,” dyed here in the red fluorescence spectrum) were integrated at the 5′ end of the endogenous FUS sequence in human induced pluripotent stem cells. Cells were screened and expanded to obtain a clonal iPSC line where GFP and Halo integrated into separate alleles of FUS. This “dual-tag” system can be used to distinguish editing of a specific allele of FUS by the loss of the fluorescent marker tagged to that allele. FIG. 6A) A schematic of the fluorescent marker tagged to each allele of FUS, the associated SNPs on that allele, and the resulting color of cells in which that allele of FUS is edited, resulting in knockout of both FUS protein and the associated tag. FIG. 6B) Dual-tagged iPSCs were electroporated with the indicated gRNA and Cas9 protein, cultured for 10 days, and imaged for fluorescence. All gRNA are capable of knocking out expression of their target FUS allele, as demonstrated by cells expressing only one tag (see “Overlay”). Dapi is a nuclear dye used to visualize all cells.

### BIBLIOGRAPHY

1. Sabatelli, M., Moncada, A., Conte, A., Lattante, S., Marangi, G., Luigetti, M., Lucchini, M., Mirabella, M., Romano, A., Del Grande, A., et al. (2013). Mutations in the 3′ untranslated region of FUS causing FUS overexpression are associated with amyotrophic lateral sclerosis. Hum. Mol. Genet. 22, 4748-4755.
2. Zou, Z.-Y., Zhou, Z.-R., Che, C.-H., Liu, C.-Y., He, R.-L., and Huang, H.-P. (2017). Genetic epidemiology of amyotrophic lateral sclerosis: a systematic review and meta-analysis. J. Neurol. Neurosurg. Psychiatr. 88, 540-549.
3. Ramos, D.M., Skarnes, W.C., Singleton, A.B., Cookson, M.R., and Ward, M.E. (2021). Tackling neurodegenerative diseases with genomic engineering: A new stem cell initiative from the NIH. Neuron 109, 1080-1083.
4. Hofmann JW, Seeley WW, Huang EJ. RNA binding proteins and the pathogenesis of frontotemporal lobar degeneration. Annu Rev Pathol. 2019 Jan 24; 14:469-95.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. A method comprising editing an endogenous FUS gene in at least one cell to reduce expression of a mutant FUS protein correlated with a neurodegenerative condition or disease.
2. The method of statement 1, wherein the neurodegenerative condition or disease is a frontotemporal dementia (FTD), an amyotrophic lateral sclerosis (ALS), or a combination thereof.
3. The method of statement 1 or 2, wherein the endogenous FUS gene is heterozygous with a mutation one FUS allele.
4. The method of statement 3, wherein the mutation in one FUS allele is a dominant mutation.
5. The method of statement 3 or 4, wherein the mutation in one FUS allele causes expression of the mutant FUS protein.
6. The method of any one of statements 3-5, wherein the mutation in one FUS allele is edited.
7. The method of any one of statements 1-6, wherein the FUS gene is edited in one or more of FUS exons 1-4.
8. The method of any one of statements 1-7, wherein the FUS gene is edited in FUS exon 3 or FUS exon 4.
9. The method of any one of statements 1-8, wherein editing the endogenous FUS gene eliminates expression of the mutant FUS protein.
10. The method of any one of statements 1-9, wherein editing the endogenous FUS gene comprises CRISPR modification of one allele of the endogenous FUS gene.
11. The method of any one of statements 1-10, wherein editing the endogenous FUS gene comprises nuclease cleavage of the endogenous FUS gene within at least one site recognized by at least one guide RNA.
12. The method of any one of statements 1-11, wherein editing the endogenous FUS gene comprises guide RNA recognition of a FUS genomic site comprising a single nucleotide polymorphism (SNP).
13. The method of any one of statements 1-12, wherein editing the endogenous FUS gene comprises guide RNA recognition of a FUS genomic site comprising a single nucleotide polymorphism (SNP) that is upstream (5′) of the mutation in the one FUS allele.
14. The method of any one of statements 11-13, wherein the guide RNA comprises an RNA sequence having at least 80%, at least 85%, at least 90%, or at least 93%, or at least 95%, or at least 96%, or at least 97%, or at least 98% sequence identity to any of SEQ ID NOs: 9-14.
15. The method of any one of statements 1-14, wherein editing the endogenous FUS gene comprises contacting at least one cell with (a) a ribonucleoprotein complex comprising at least one nuclease and at least one guide RNA; or (b) an expression system comprising at least one an expression cassette comprising a promoter operably linked to a nucleic acid segment encoding at least one guide RNA, at least one nuclease, or a combination of at least one guide RNA and at least one nuclease.
16. The method of statement 15, wherein the at least one cell is a population of cells.
17. The method of statement 15 or 16, wherein the at least one cell is maintained in vitro in a culture medium.
18. The method of any one of statements 1-17, wherein the at least one cell is allogeneic or autologous to a subject suspected of having the neurodegenerative condition or disease.
19. The method of any one of statements 1-18, wherein the at least one cell is a neuronal cell, neuronal tissue, or a population of neuronal cells.
20. The method of any one of statements 15-19, further comprising confirming that expression of the mutant FUS protein correlated with a neurodegenerative condition or disease is reduced or eliminated in the at least one cell, to thereby identify at least one cell with a corrected FUS gene.
21. The method of statement 20, further comprising administering the at least one cell with the corrected FUS gene to a subject.
22. The method of any one of statements 15-19, wherein the at least one cell is in vivo within a subject, and the subject is suspected of having the neurodegenerative condition or disease.
23. The method of statement 22, wherein contacting at least one cell comprises (a) administering at least one guide RNA and a nuclease to the subject; or (b) administering to the subject an expression system comprising at least one an expression cassette comprising a promoter operably linked to a nucleic acid segment encoding at least one guide RNA, at least one nuclease, or a combination of at least one guide RNA and at least one nuclease.
24. The method of statement 23, comprising administering a ribonucleoprotein complex comprising the at least one guide RNA and the nuclease to the subject.
25. The method of statement 22 or 23, comprising locally administering a ribonucleoprotein complex comprising the at least one guide RNA and the nuclease to the subject.
26. The method of any one of statements 22-25, comprising administering a ribonucleoprotein complex comprising the at least one guide RNA and the nuclease to neuronal cell, neuronal tissue, or a population of neuronal cells in the subject.
27. A guide RNA comprising an RNA sequence having at least 80%, at least 85%, at least 90%, or at least 93%, or at least 95%, or at least 96%, or at least 97%, or at least 98% sequence identity to any of SEQ ID NOs: 9-14.
28. The guide RNA of statement 27, wherein each of the guide RNAs includes a Protospacer Adjacent Motif (PAM) sequence.
29. A method comprising administering the guide RNA of statement 27 or 28 to a cell or to a subject.
30. The method of statement 29, comprising administering the guide RNA of statement 27 or 28 to neuronal tissues or neuronal cells.
31. A cell or population of cells comprising the guide RNA of statement 27 or 28.
32. A method comprising administering the cell or the population of cells of statement 31 to a subject.
33. The method of statement 32, comprising administering the cell or the population of cells of statement 31 to neuronal tissues or neuronal cells.
34. A composition comprising a carrier and one or more of the guide RNAs with an RNA sequence having at least 80%, at least 85%, at least 90%, or at least 93%, or at least 95%, or at least 96%, or at least 97%, or at least 98% sequence identity to any of SEQ ID NOs: 9-14.
35. The composition of claim 34, further comprising a nuclease.
36. The composition of claim 35, wherein the nuclease is a Streptococcus pyogenes Cas9, (SpCas9), Staphylococcus aureus Cas9 (SaCas9), a Francisella novicida Cas2 or a combination thereof.
37. A method comprising administering the composition of any one of statements 34-36 to a cell or to a subject.
38. The method of statement 37, comprising administering the composition of any one of statements 34-36 to neuronal tissues or neuronal cells.
39. A cell or population of cells comprising the composition of any of statements 34-36.
40. The cell or population of cells of statement 39, which are neuronal cells.
41. A method comprising administering the cell or population of cells of statement 39 or 40 to a subject.
42. A ribonucleoprotein complex comprising at least one nuclease and at least one guide RNA comprising an RNA sequence having at least 80%, at least 85%, at least 90%, or at least 93%, or at least 95%, or at least 96%, or at least 97%, or at least 98% sequence identity to any of SEQ ID NOs: 9-14.
43. The ribonucleoprotein complex of statement 42, wherein the each of the guide RNAs includes a Protospacer Adjacent Motif (PAM) sequence.
44. The ribonucleoprotein complex of statement 42 or 43, wherein the nuclease is a Cas9 nuclease.
45. The ribonucleoprotein complex of any one of statements 42-44, wherein the nuclease is a Streptococcus pyogenes Cas9, (SpCas9), Staphylococcus aureus Cas9 (SaCas9), a Francisella novicida Cas2 or a combination thereof.
46. A method comprising administering the ribonucleoprotein complex of any one of statements 42-45 to a cell or to a subject.
47. The method of statement 46, wherein the cell is a neuronal cell or a neuronal tissue.
48. A cell or population of cells comprising the ribonucleoprotein complex of any of statements 42-46.
49. A method comprising administering the cell or the population of cells of statement 48 to a subject.
50. An expression system comprising one or more expression cassettes or expression vectors comprising a first promoter operably linked to a nucleic acid segment with at least 80%, at least 85%, at least 90%, or at least 93%, or at least 95%, or at least 96%, or at least 97%, or at least 98% sequence identity to any of SEQ ID NOs: 9-14.
51. The expression system of statement 50, wherein the first promoter is an inducible promoter, a tissue-specific promoter, or a cell-type specific promoter.
52. The expression system of statement 50 or 51, further comprising an expression cassette or expression vector comprising a second promoter operably linked to a nucleic acid segment encoding a nuclease.
53. The expression system of statement 52, wherein the nuclease is a Streptococcus pyogenes Cas9, (SpCas9), Staphylococcus aureus Cas9(SaCas9), a Francisella novicida Cas2 or a combination thereof.
54. A method comprising administering the expression system of any of statements 50-53 to a cell or to a subject.
55. The method of statement 54, wherein the cell is a neuronal cell or a neuronal tissue.
56. A cell or population of cells comprising the expression system of any of statements 50-53.
57. A method comprising administering the cell or the population of cells of statement 56 to a subject.
58. The method of statement 57, wherein the cell or the population of cells are administered to neuronal tissues of the subject.

The specific methods and compositions described herein are representative of embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed:

1. A method comprising editing an endogenous FUS gene in at least one cell to reduce expression of a mutant FUS protein correlated with a neurodegenerative condition or disease.

2. The method of claim 1, wherein the neurodegenerative condition or disease is a frontotemporal dementia (FTD), an amyotrophic lateral sclerosis (ALS), or a combination thereof.

3. The method of claim 1, wherein the endogenous FUS gene is heterozygous with a mutation in one FUS allele.

4. The method of claim 3, wherein the mutation in one FUS allele is a dominant mutation.

5. The method of claim 3, wherein the mutation in one FUS allele causes expression of the mutant FUS protein.

6. The method of claim 3, wherein the mutation in one FUS allele is edited.

7. The method of claim 1, wherein the FUS gene is edited in one or more of FUS exons 1-4.

8. The method of claim 1, wherein the FUS gene is edited in FUS exon 3 or FUS exon 4.

9. The method of claim 1, wherein editing the endogenous FUS gene eliminates expression of the mutant FUS protein.

10. The method of claim 1, wherein editing the endogenous FUS gene comprises CRISPR modification of one allele of the endogenous FUS gene.

11. The method of claim 1, wherein editing the endogenous FUS gene comprises nuclease cleavage of the endogenous FUS gene within at least one site recognized by at least one guide RNA.

12. The method of claim 1, wherein editing the endogenous FUS gene comprises guide RNA recognition of a FUS genomic site comprising a single nucleotide polymorphism (SNP).

13. The method of claim 1, wherein editing the endogenous FUS gene comprises guide RNA recognition of a FUS genomic site comprising a single nucleotide polymorphism (SNP) that is upstream (5′) of the mutation in the one FUS allele.

14. The method of claim 11, wherein the guide RNA comprises an RNA sequence having at least 80%, at least 85%, at least 90%, or at least 93%, or at least 95%, or at least 96%, or at least 97%, or at least 98% sequence identity to any of SEQ ID NOs: 9-14.

15. The method of claim 1, wherein editing the endogenous FUS gene comprises contacting at least one cell with (a) a ribonucleoprotein complex comprising at least one nuclease and at least one guide RNA; or (b) an expression system comprising at least one an expression cassette comprising a promoter operably linked to a nucleic acid segment encoding at least one guide RNA, at least one nuclease, or a combination of at least one guide RNA and at least one nuclease.

16. The method of claim 15, wherein the at least one cell is a population of cells.

17. The method of claim 15, wherein the at least one cell is maintained in vitro in a culture medium.

18. The method of claim 1, wherein the at least one cell is allogeneic or autologous to a subject suspected of having the neurodegenerative condition or disease.

19. The method of claim 1, wherein the at least one cell is a neuronal cell, neuronal tissue, or a population of neuronal cells.

20. The method of claim 15, further comprising confirming that expression of the mutant FUS protein correlated with a neurodegenerative condition or disease is reduced or eliminated in the at least one cell, to thereby identify at least one cell with a corrected FUS gene.

21. The method of claim 20, further comprising administering the at least one cell with the corrected FUS gene to a subject.

22. The method of claim 15, wherein the at least one cell is in vivo within a subject, and the subject is suspected of having the neurodegenerative condition or disease.

23. The method of claim 22, wherein contacting at least one cell comprises (a) administering at least one guide RNA and a nuclease to the subject; or (b) administering to the subject an expression system comprising at least one an expression cassette comprising a promoter operably linked to a nucleic acid segment encoding at least one guide RNA, at least one nuclease, or a combination of at least one guide RNA and at least one nuclease.

24. The method of claim 23, comprising administering a ribonucleoprotein complex comprising the at least one guide RNA and the nuclease to the subject.

25. The method of claim 22, comprising locally administering a ribonucleoprotein complex comprising the at least one guide RNA and the nuclease to the subject.

26. The method of claims 22, comprising administering a ribonucleoprotein complex comprising the at least one guide RNA and the nuclease to neuronal cell, neuronal tissue, or a population of neuronal cells in the subject.

27. A guide RNA comprising an RNA sequence having at least 80%, at least 85%, at least 90%, or at least 93%, or at least 95%, or at least 96%, or at least 97%, or at least 98% sequence identity to any of SEQ ID NOs: 9-14.

28. The guide RNA of claim 27, wherein each of the guide RNAs includes a Protospacer Adjacent Motif (PAM) sequence.

29. A method comprising administering the guide RNA of claim 27 to a cell or to a subject.

30. The method of claim 29, comprising administering the guide RNA of claim 27 to neuronal tissues or neuronal cells.

31. A cell or population of cells comprising the guide RNA of claim 27.

32. A method comprising administering the cell or the population of cells of claim 31 to a subject.

33. The method of claim 32, comprising administering the cell or the population of cells of claim 31 to neuronal tissues or neuronal cells.

34. A composition comprising a carrier and one or more of the guide RNAs with an RNA sequence having at least 80%, at least 85%, at least 90%, or at least 93%, or at least 95%, or at least 96%, or at least 97%, or at least 98% sequence identity to any of SEQ ID NOs: 9-14.

35. The composition of claim 34, further comprising a nuclease.

36. The composition of claim 35, wherein the nuclease is a Streptococcus pyogenes Cas9, (SpCas9), Staphylococcus aureus Cas9 (SaCas9), a Francisella novicida Cas2 or a combination thereof.

37. A method comprising administering the composition of claims 34 to a cell or to a subject.

38. The method of claim 37, comprising administering the composition of claim 34 to neuronal tissues or neuronal cells.

39. A cell or population of cells comprising the composition of claim 34.

40. The cell or population of cells of claim 39, which are neuronal cells.

41. A method comprising administering the cell or population of cells of claim 39 to a subject.

42. A ribonucleoprotein complex comprising at least one nuclease and at least one guide RNA comprising an RNA sequence having at least 80%, at least 85%, at least 90%, or at least 93%, or at least 95%, or at least 96%, or at least 97%, or at least 98% sequence identity to any of SEQ ID NOs: 9-14.

43. The ribonucleoprotein complex of claim 42, wherein the each of the guide RNAs includes a Protospacer Adjacent Motif (PAM) sequence.

44. The ribonucleoprotein complex of claim 42, wherein the nuclease is a Cas9 nuclease.

45. The ribonucleoprotein complex of claim 42, wherein the nuclease is a Streptococcus pyogenes Cas9, (SpCas9), Staphylococcus aureus Cas9 (SaCas9), a Francisella novicida Cas2 or a combination thereof.

46. A method comprising administering the ribonucleoprotein complex of claim 42 to a cell or to a subject.

47. The method of claim 46, wherein the cell is a neuronal cell or a neuronal tissue.

48. A cell or population of cells comprising the ribonucleoprotein complex of claim 42.

49. A method comprising administering the cell or the population of cells of claim 48 to a subject.

50. An expression system comprising one or more expression cassettes or expression vectors comprising a first promoter operably linked to a nucleic acid segment with at least 80%, at least 85%, at least 90%, or at least 93%, or at least 95%, or at least 96%, or at least 97%, or at least 98% sequence identity to any of SEQ ID NOs: 9-14.

51. The expression system of claim 50, wherein the first promoter is an inducible promoter, a tissue-specific promoter, or a cell-type specific promoter.

52. The expression system of claim 50, further comprising an expression cassette or expression vector comprising a second promoter operably linked to a nucleic acid segment encoding a nuclease.

53. The expression system of claim 52, wherein the nuclease is a Streptococcus pyogenes Cas9, (SpCas9), Staphylococcus aureus Cas9 (SaCas9), a Francisella novicida Cas2 or a combination thereof.

54. A method comprising administering the expression system of claim 50 to a cell or to a subject.

55. The method of claim 54, wherein the cell is a neuronal cell or a neuronal tissue.

56. A cell or population of cells comprising the expression system of claim 50.

57. A method comprising administering the cell or the population of cells of claim 56 to a subject.

58. The method of claim 57, wherein the cell or the population of cells are administered to neuronal tissues of the subject.
